(19) United States
(12) Patent Application Publication
(10) Pub. No.: US 2009/0275548 A1
(43) Pub. Date: Nov. 5, 2009

(54) COMPOUNDS AND METHODS FOR ACTIVATED THERAPY

(76) Inventors: Thomas Lewis, Winchester, MA (US); Donald J. Burke, Boston, MA (US); Ann M. Szulwach, Middle Village, NY (US)

Correspondence Address: ELMORE PATENT LAW GROUP, PC, 515 Groton Road, Unit 1R, Westford, MA, 01886, US

(21) Appl. No.: 12/424,383

(22) Filed: Apr. 15, 2009

Related U.S. Application Data
(60) Provisional application No. 61/046,098, filed on Apr. 18, 2008.

(51) Int. Cl. A61K 31/555; A61P 35/00; A61P 31/12; A61P 31/04; A61P 9/00; A61P 25/00

(52) U.S. Cl. 514/185

(57) ABSTRACT

Provided herein are compounds for detection, diagnosis and treatment of target tissues or target compositions, including hyperproliferative tissues such as tumors, using sonodynamic and/or photodynamic methods. In particular, photosensitizer and/or sonosensitizer compounds that collect in hyperproliferative tissue are provided.

### RELATED APPLICATION

This application claims the benefit of U.S. Provisional Application No. 61/046,098, filed on Apr. 16, 2008. The entire teaching of the above application is incorporated herein by reference.

### BACKGROUND OF THE INVENTION

Porphyrins, the so-called “expanded porphyrins”, and related polypyrrole structures are members of a class of macrocycles capable of forming stable complexes with metals. The metal is constrained (as its cation) within a central binding cavity of the macrocycle (the “core”). The anions associated with the metal cation are found above and below the core; and are called apical ligands. Examples of this class of macrocycles are porphyrins, porphyrin isomers, porphyrin-like macrocycles, benzoporphyrins, texaphyrins, alaskaphyrins, sapphyrins, rubyrins, porphycenes, chlorins, benzochlorins, bacteriochlorins, and purpurins. Examples of metalized prophyrins are chlorophyll where the metal is Mg(II), Vitamin B12 where the metal is Co(II), and Heme where the metal is Fe(II).

Porphyrins have been suggested to be useful as photosensitizers in photodynamic therapy in oncology and other disease states. In photodynamic therapy, a photosensitizer compound that demonstrates the ability to selectively accumulate in target tissue, such as neoplastic or hyperproliferative tissue, is administered to a subject, and when the photosensitizer accumulates in or preferentially associates with the target tissue, the target tissue becomes sensitized to photoradiation. The photo-sensitizing agent can be activated either by coherent (laser) or non-coherent (non-laser) light. It is currently accepted that following absorption of light, the photosensitizer is transformed from its ground singlet state (P) into an electronically excited triplet state (³P*; T˜10⁻² sec.) via a short-lived excited singlet state (¹P*; T˜10⁻⁶ sec.). The excited triplet can undergo non-radiative decay or participate in an electron transfer process with biological substrates to form radicals and radical ions, which can produce singlet oxygen and superoxide (O₂⁻) after interaction with molecular oxygen (O₂). Singlet oxygen can be produced from molecular oxygen by the transfer of energy directly or indirectly from the activated photosensitizer. Singlet oxygen is one of the agents responsible for cellular and tissue damage in PDT, causing oxidation of the target tissue; there also is evidence that the superoxide ion may be involved. The generation of these cytotoxic agents plays a role in tumor homeostasis and the observed tumor destruction.

In PDT, a photosensitizing agent (“photosensitizer”) is delivered to the target tissue and then radiation, most usually light of wavelengths between 250-1000 nm, e.g., 500-800 nm, or 600-700 nm, is applied to the target tissue. Thus, photosensitizing agents are activated by electromagnetic (“EM”) radiation.

In addition, select compounds can also be activated by acoustic energy, in particular ultrasonic or sonic energy (see, e.g., Misik and Riesz, Ann. N.Y. Acad. Sci. 899:335-48 (2000); and U.S. Pat. No. 5,817,048) in a process known as sonosensitization. When sonosensitization is applied in a therapeutic mode, it is referred to as Sonodynamic Therapy (“SDT”) and more recently also referred to as Ultrasound Activated Therapy (“USAT”), Activated Cancer Therapy (ACT), the Sonnelux Protocol, and the Sonnelux treatment.

Despite recent advances in PDT and SDT, there is still a need for additional sensitizers, including photosensitizers, sonosensitizers and dual acting (photo- and sono) sensitizers for use in SDT, PDT, USAT, ACT, diagnostic and therapeutic applications.

### SUMMARY OF THE INVENTION

The invention is based on the unexpected discovery of novel sonosensitizers having optimized sonodynamic properties. The sensitizers have low toxicity prior to activation as determined in LC50 studies; target areas of tumor growth and activity including: tumor cells, tumor cell membranes or the neovascular network of the tumor; are rapidly cleared from non-tumor compartments of the body; are highly sensitive to activation using readily available and safe ultrasound frequencies and intensities and red light; and have minimal side effects to the body systemically or to the local area upon activation.

Accordingly, the present invention provides methods and compounds having a general formula I:

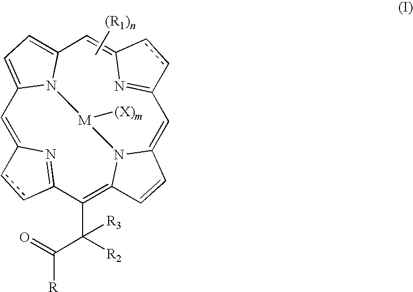

or its geometric isomers, enantiomers, diastereomers, racemates, pharmaceutically acceptable salts, prodrugs and solvates thereof, wherein R is OR₄ or NR₄R₅ each R₄ and R₅ is independently selected from hydrogen, a substituted or unsubstituted, saturated or unsaturated alkyl group or a substituted or unsubstituted aryl group or other blocking or protective group; alternatively, R₄ and R₅ can be taken together with the nitrogen they are attached to form a substituted or unsubstituted heterocyclic group;

each R₁ is independently selected from a substituted or unsubstituted, saturated or unsaturated alkyl group, a substituted or unsubstituted aryl group, acid, ester, amide, amine, substituted amine, acyl, hydroxy, ether, halogen, nitrile, aldehyde, thiol, thioether, sulfonic acid, sulfonate, sulfonamide, and sulfate;

R₂ and R₃ are independently selected from hydrogen, a substituted or unsubstituted, saturated or unsaturated alkyl group;

n is zero or an integer from 1 to 10;

each [symbol] represents a single or double bond;

M represents a metal at oxidation state I-VII, preferably tin (Sn);

X is selected from the group consisting of anions, acids (acetate, for example) F, Cl, Br, I, H, CN, a substituted or unsubstituted hydroxide group, a substituted or unsubstituted amino group, a substituted or unsubstituted, straight or branched C₁-C₂₀ alkyl group, an acyl group, a thiolate group or a dialkylamino group; preferably OH and/or acetate are preferred and m represents 2, 3, 4 or 5 and is chosen to maintain the electric neutrality of the metal complex compound.

In a preferred embodiment of the invention relates to chlorin derivatives, such as metallated chlorin-e6 and derivatives thereof, including its ester or amide derivatives, have sonosensitizing properties and may be used to treat diseases and other conditions in humans and animals. Moreover, the CE6 derivatives of the present invention may be modified, derivatized and/or conjugated to a delivery moiety to enhance the ability of the derivative to target predetermined cells or structures in vitro or in vivo.

The methods and compositions of the present invention, activated by sound and/or light, exhibit substantial absorption in the therapeutic frequencies of ultrasound and/or red light; produce high cytotoxic component yield; can be produced in pure, monomeric form; may be derivatized, modified and/or conjugated to optimize properties of ultrasound activation and/or light activation, tissue biodistribution, and toxicity; and are rapidly cleared and excreted. They afford tumor targeting by covalent or physical attachment to cell membranes or penetrate into the cells to enhance sonotoxicity and phototoxicity.

The invention further provides compounds for use in SDT, PDT, SPDT combination therapy, USAT, ACT, diagnostic and therapeutic applications. In one embodiment, the compounds preferentially absorb into target tissue, including hyperproliferative tissue. Diseases and conditions which can be treated include cancer, tumors, infectious diseases, inflammatory diseases, autoimmune diseases, cardiovascular diseases, neurodegenerative diseases, and granulomatous diseases.

### BRIEF DESCRIPTION OF THE DRAWINGS

FIG. 1 illustrates a Lethality curve for SF-2;

FIG. 2 is a dose-response curve of SF-2 cytotoxicity of human melanoma cancer cell. X axis represents SF-2 concentration, Y axis represents the % Cell death when compared to the vehicle control. Each points represents mean±SE (n=6);

FIG. 3 illustrates a Lethality curve for SF-1;

FIG. 4 represents standard curves used to calculate cell number in each group. The equation on each graph was used to calculate cell number for each condition; y=0.0124X+1620 was used for RFU >5730, y=0.022X+397.74 was used for RFU ≦5730. X axis represents cell number, Y axis represents RFU measurement;

FIG. 5 is a dose-response curve of SF1 cytotoxicity of human melanoma cancer cell. X axis represents SF1 concentration, Y axis represents the % Cell death when compared to the vehicle control. Each points represents mean±SE (n=6); and

FIG. 6 shows the absorption spectrum for SF1.

### DETAILED DESCRIPTION OF THE INVENTION

In a first embodiment of the compounds of the present invention are compounds represented by formula as illustrated above, or its geometric isomers, enantiomers, diastereomers, racemates, pharmaceutically acceptable salts, prodrugs and solvates thereof.

In one embodiment of the compounds of the present invention are compounds represented by formula II as illustrated below, or its geometric isomers, enantiomers, diastereomers, racemates, pharmaceutically acceptable salts, prodrugs and solvates thereof:

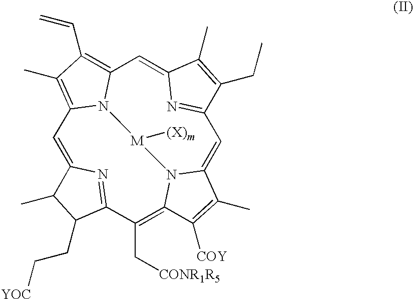

wherein Y is hydroxy, substituted hydroxy, prodrug group or an acceptable metal salt. In one example NR₄R₅ are amino acid, amino acid derivative, or peptide. Amino acid derivatives are those derived from valine, leucine, isoleucine, threonine, methionine, phenylalanine, tryptophan, alanine, arginine, aspartic acid, cysteine, cysteine, glutamic acid, glycine, histidine, proline, serine, tyrosine, asparagines, and glutamine. Amino acid-like derivatives are also included, but are not limited to taurine. Also useful are peptides, particularly those known to have affinity for specific receptors, including but not limited to, oxytocin, vasopressin, bradykinin, LHRH, thrombin, and the like. In another example, NR₄R₅ represents an amine terminated polyethylene glycol (PEG). PEGylation provides for improved bioavailability, including longer circulation time and slower clearance. In particular, it improves the delivery of injectable proteins as well as other compounds. It can also be used for controlled agent release and optimized pharmacokinetics resulting in sustained duration. In addition, PEGylation improves the safety profile with lower toxicity, immunogenicity, and antigenicity. It can also provide increased efficacy and decreased dosing frequency. PEGylation also improves agent solubility and stability and reduces susceptibility to proteolysis. The PEGs are selected from a broad range of molecular weights (5-60 kDa), functional groups, and attachment chemistries. For example, PEG can be 12-40 kDa, it can be branched or unbranched, binding can be through an —NHS reactive group, and binding sites can include lysine or histidine residues. In one example R₄ is hydrogen and R₅ is a —(CH₂CH₂O)rCH₂CH₂OH wherein r is an integer between 1 and 100.

In one embodiment of the compounds of the present invention are compounds represented by formula III as illustrated below, or its geometric isomers, enantiomers, diastereomers, racemates, pharmaceutically acceptable salts, prodrugs and solvates thereof:

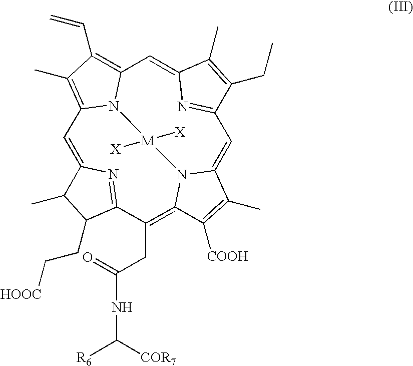

wherein R₆ is hydrogen, a substituted or unsubstituted, saturated or unsaturated alkyl, substituted or unsubstituted aryl;

R₇ and is a hydroxy, substituted hydroxy, amine or substituted amine;

M and X are as previously defined.

In one embodiment, when M is at oxidation state IV, then m=2; some examples are Ti(IV), Zr(IV), Hf(IV), Sn(IV), Mo(IV), V(IV) and W(IV). When M is at oxidation state V, then m=3; some examples are V(V), Nb(V) and Ta(V). When M is at oxidation state VI, then m=4; some examples are Cr(VI), Mo(VI), W(VI) and Re(VI). When M is at oxidation state VII, then m=5; some examples are Tc(VII) and Re(VII). It is preferable to use metal complex compounds of general formula (I) with metals M at oxidation state IV. Preferably M at oxidation state IV includes Si(IV), Ti(IV), Sn(IV), Zr(IV), Hf(IV), Th(IV), Sn (IV), Mo(IV), V(IV) and W(IV). Sn(IV) are particularly preferred.

Compounds according to the invention include compounds of formula:

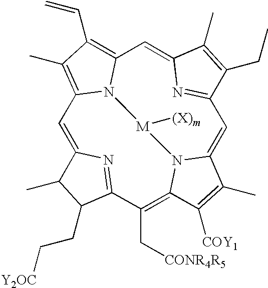

wherein M, Y₁, Y₂, and NR₄R₅ are set forth in the table below:

TABLE A

| Compound | | | | |
|---|---|---|---|---|
| No. | M | Y₁ | Y2 | —NR₄R₅ |
| 1 | Sn(IV) | OH | OH | —NHCH₂COOH |
| 2 | Sn(IV) | OMe | OH | —NHCH₂COOH |
| 3 | Sn(IV) | OH | OMe | —NHCH₂COOH |
| 4 | Sn(IV) | OEt | OH | —NHCH₂COOH |
| 5 | Sn(IV) | OH | OEt | —NHCH₂COOH |
| 6 | Sn(IV) | NH₂ | OH | —NHCH₂COOH |
| 7 | Sn(IV) | OH | NH₂ | —NHCH₂COOH |
| 8 | Sn(IV) | OH | OH | —NHCHCH₃COOH |
| 9 | Sn(IV) | OMe | OH | —NHCHCH₃COOH |
| 10 | Sn(IV) | OH | OMe | —NHCHCH₃COOH |
| 11 | Sn(IV) | OEt | OH | —NHCHCH₃COOH |
| 12 | Sn(IV) | OH | OEt | —NHCHCH₃COOH |
| 13 | Sn(IV) | NH₂ | OH | —NHCHCH₃COOH |
| 14 | Sn(IV) | OH | NH₂ | —NHCHCH₃COOH |
| 15 | Sn(IV) | OH | OH | —NHCHC₂H₅COOH |
| 16 | Sn(IV) | OMe | OH | —NHCHC₂H₅COOH |
| 17 | Sn(IV) | OH | OMe | —NHCHC₂H₅COOH |
| 18 | Sn(IV) | OEt | OH | —NHCHC₂H₅COOH |
| 19 | Sn(IV) | OH | OEt | —NHCHC₂H₅COOH |
| 20 | Sn(IV) | NH₂ | OH | —NHCHC₂H₅COOH |
| 21 | Sn(IV) | OH | NH₂ | —NHCHC₂H₅COOH |
| 22 | Sn(IV) | OH | OH | —NHCH(CHPh)COOH |
| 23 | Sn(IV) | OMe | OH | —NHCH(CHPh)COOH |
| 24 | Sn(IV) | OH | OMe | —NHCH(CHPh)COOH |
| 25 | Sn(IV) | OEt | OH | —NHCH(CHPh)COOH |
| 26 | Sn(IV) | OH | OEt | —NHCH(CHPh)COOH |
| 27 | Sn(IV) | NH₂ | OH | —NHCH(CHPh)COOH |
| 28 | Sn(IV) | OH | NH₂ | —NHCH(CHPh)COOH |
| 29 | Sn(IV) | OH | OH | —NHCH(CHOH)COOH |
| 30 | Sn(IV) | OMe | OH | —NHCH(CHOH)COOH |
| 31 | Sn(IV) | OH | OMe | —NHCH(CHOH)COOH |
| 32 | Sn(IV) | OEt | OH | —NHCH(CHOH)COOH |
| 33 | Sn(IV) | OH | OEt | —NHCH(CHOH)COOH |
| 34 | Sn(IV) | NH₂ | OH | —NHCH(CHOH)COOH |
| 35 | Sn(IV) | OH | NH₂ | —NHCH(CHOH)COOH |
| 36 | Sn(IV) | OH | OH | —NHCH(CH₂COOH)COOH |
| 37 | Sn(IV) | OMe | OH | —NHCH(CH₂COOH)COOH |
| 38 | Sn(IV) | OH | OMe | —NHCH(CH₂COOH)COOH |
| 39 | Sn(IV) | OEt | OH | —NHCH(CH₂COOH)COOH |
| 40 | Sn(IV) | OH | OEt | —NHCH(CH₂COOH)COOH |
| 41 | Sn(IV) | NH₂ | OH | —NHCH(CH₂COOH)COOH |
| 42 | Sn(IV) | OH | NH₂ | —NHCH(CH₂COOH)COOH |
| 43 | Sn(IV) | OH | OH | —NHCH(CH₂CH₂COOH)COOH |
| 44 | Sn(IV) | OMe | OH | —NHCH(CH₂CH₂COOH)COOH |
| 45 | Sn(IV) | OH | OMe | —NHCH(CH₂CH₂COOH)COOH |
| 46 | Sn(IV) | OEt | OH | —NHCH(CH₂CH₂COOH)COOH |
| 47 | Sn(IV) | OH | OEt | —NHCH(CH₂CH₂COOH)COOH |
| 48 | Sn(IV) | NH₂ | OH | —NHCH(CH₂CH₂COOH)COOH |
| 49 | Sn(IV) | OH | NH₂ | —NHCH(CH₂CH₂COOH)COOH |
| 50 | Sn(IV) | OH | OH | —HNCH((CH₂)₄NH₂)COOH |
| 51 | Sn(IV) | OMe | OH | —HNCH((CH₂)₄NH₂)COOH |
| 52 | Sn(IV) | OH | OMe | —HNCH((CH₂)₄NH₂)COOH |
| 53 | Sn(IV) | OEt | OH | —HNCH((CH₂)₄NH₂)COOH |
| 54 | Sn(IV) | OH | OEt | —HNCH((CH₂)₄NH₂)COOH |
| 55 | Sn(IV) | NH₂ | OH | —HNCH((CH₂)₄NH₂)COOH |
| 56 | Sn(IV) | OH | NH₂ | —HNCH((CH₂)₄NH₂)COOH |
| 57 | Sn(IV) | OH | OH | —HNCH((CH₂)₃NH₂)COOH |
| 58 | Sn(IV) | OMe | OH | —HNCH((CH₂)₃NH₂)COOH |
| 59 | Sn(IV) | OH | OMe | —HNCH((CH₂)₃NH₂)COOH |
| 60 | Sn(IV) | OEt | OH | —HNCH((CH₂)₃NH₂)COOH |
| 61 | Sn(IV) | OH | OEt | —HNCH((CH₂)₃NH₂)COOH |
| 62 | Sn(IV) | NH₂ | OH | —HNCH((CH₂)₃NH₂)COOH |
| 63 | Sn(IV) | OH | NH₂ | —HNCH((CH₂)₃NH₂)COOH |
| 64 | Sn(IV) | OH | OH | —HNCH((CH₂)₃NH(CNH₂)═NH)COOH |
| 65 | Sn(IV) | OMe | OH | —HNCH((CH₂)₃NH(CNH₂)═NH)COOH |
| 66 | Sn(IV) | OH | OMe | —HNCH((CH₂)₃NH(CNH₂)═NH)COOH |
| 67 | Sn(IV) | OEt | OH | —HNCH((CH₂)₃NH(CNH₂)═NH)COOH |
| 68 | Sn(IV) | OH | OEt | —HNCH((CH₂)₃NH(CNH₂)═NH)COOH |
| 69 | Sn(IV) | NH₂ | OH | —HNCH((CH₂)₃NH(CNH₂)═NH)COOH |
| 70 | Sn(IV) | OH | NH₂ | —HNCH((CH₂)₃NH(CNH₂)═NH)COOH |
| 71 | Sn(IV) | OH | OH | —HNCH(CH₂CONH₂)COOH |
| 72 | Sn(IV) | OMe | OH | —HNCH(CH₂CONH₂)COOH |
| 73 | Sn(IV) | OH | OMe | —HNCH(CH₂CONH₂)COOH |
| 74 | Sn(IV) | OEt | OH | —HNCH(CH₂CONH₂)COOH |
| 75 | Sn(IV) | OH | OEt | —HNCH(CH₂CONH₂)COOH |
| 76 | Sn(IV) | NH₂ | OH | —HNCH(CH₂CONH₂)COOH |
| 77 | Sn(IV) | OH | NH₂ | —HNCH(CH₂CONH₂)COOH |
| 78 | Sn(IV) | OH | OH | —HNCH(CH₂CH₂CONH₂)COOH |
| 79 | Sn(IV) | OMe | OH | —HNCH(CH₂CH₂CONH₂)COOH |
| 80 | Sn(IV) | OH | OMe | —HNCH(CH₂CH₂CONH₂)COOH |
| 81 | Sn(IV) | OEt | OH | —HNCH(CH₂CH₂CONH₂)COOH |
| 82 | Sn(IV) | OH | OEt | —HNCH(CH₂CH₂CONH₂)COOH |
| 83 | Sn(IV) | NH₂ | OH | —HNCH(CH₂CH₂CONH₂)COOH |
| 84 | Sn(IV) | OH | OH | —(NHCH₂CO)₂OH |
| 85 | Sn(IV) | OH | OH | —(NHCH₂CO)₃OH |
| 86 | Sn(IV) | OH | OH | —(NHCH₂CO)₄OH |
| 87 | Sn(IV) | OH | OH | —(NHCH₂CO)₅OH |
| 88 | Sn(IV) | OH | OH | —(NHCH₂CO)₆OH |
| 89 | Sn(IV) | OH | OH | —(NHCHCH₃CO)₄OH |
| 90 | Sn(IV) | OH | OH | —(NHCH(CHOH)CO)₄OH |
| 91 | Sn(IV) | OH | OH | —(NHCH((CH₂)₃NH₂)CO)₂OH |
| 92 | Sn(IV) | OH | OH | —(NHCH((CH₂)₃NH₂)CO)₃OH |
| 93 | Sn(IV) | OH | OH | —(NHCH((CH₂)₃NH₂)CO)₄OH |
| 94 | Sn(IV) | OH | OH | —(NHCH(CH₂CONH₂)CO)₂OH |
| 95 | Sn(IV) | OH | OH | —N(histidine) |
| 96 | Sn(IV) | OH | OH | —N(proline) |
| 97 | Sn(IV) | OH | OH | —NH(CH₂CH₂0)ₙOH |
| 98 | Sn(IV) | OH | OH | —N(folate) |
| 99 | Sn(IV) | OH | OMe | —NH(CH₂CH₂0)ₙOH |
| 100 | Sn(IV) | OMe | OH | —NH(CH₂CH₂0)ₙOH |
| 101 | Ti(IV) | OH | OH | —NHCH₂COOH |
| 102 | Ti(IV) | OMe | OH | —NHCH₂COOH |
| 103 | Ti(IV) | OH | OMe | —NHCH₂COOH |
| 104 | Ti(IV) | OEt | OH | —NHCH₂COOH |
| 105 | Ti(IV) | OH | OEt | —NHCH₂COOH |
| 106 | Ti(IV) | NH₂ | OH | —NHCH₂COOH |
| 107 | Ti(IV) | OH | NH₂ | —NHCH₂COOH |
| 108 | Ti(IV) | OH | OH | —NHCHCH₃COOH |
| 109 | Ti(IV) | OMe | OH | —NHCHCH₃COOH |
| 110 | Ti(IV) | OH | OMe | —NHCHCH₃COOH |
| 111 | Ti(IV) | OEt | OH | —NHCHCH₃COOH |
| 112 | Ti(IV) | OH | OEt | —NHCHCH₃COOH |
| 113 | Ti(IV) | NH₂ | OH | —NHCHCH₃COOH |
| 114 | Ti(IV) | OH | NH₂ | —NHCHCH₃COOH |
| 115 | Ti(IV) | OH | OH | —NHCHC₂H₅COOH |
| 116 | Ti(IV) | OMe | OH | —NHCHC₂H₅COOH |
| 117 | Ti(IV) | OH | OMe | —NHCHC₂H₅COOH |
| 118 | Ti(IV) | OEt | OH | —NHCHC₂H₅COOH |
| 119 | Ti(IV) | OH | OEt | —NHCHC₂H₅COOH |
| 120 | Ti(IV) | NH₂ | OH | —NHCHC₂H₅COOH |
| 121 | Ti(IV) | OH | NH₂ | —NHCHC₂H₅COOH |
| 122 | Ti(IV) | OH | OH | —NHCH(CHPh)COOH |
| 123 | Ti(IV) | OMe | OH | —NHCH(CHPh)COOH |
| 124 | Ti(IV) | OH | OMe | —NHCH(CHPh)COOH |
| 125 | Ti(IV) | OEt | OH | —NHCH(CHPh)COOH |
| 126 | Ti(IV) | OH | OEt | —NHCH(CHPh)COOH |
| 127 | Ti(IV) | NH₂ | OH | —NHCH(CHPh)COOH |
| 128 | Ti(IV) | OH | NH₂ | —NHCH(CHPh)COOH |
| 129 | Ti(IV) | OH | OH | —NHCH(CHOH)COOH |
| 130 | Ti(IV) | OMe | OH | —NHCH(CHOH)COOH |
| 131 | Ti(IV) | OH | OMe | —NHCH(CHOH)COOH |
| 132 | Ti(IV) | OEt | OH | —NHCH(CHOH)COOH |
| 133 | Ti(IV) | OH | OEt | —NHCH(CHOH)COOH |
| 134 | Ti(IV) | NH₂ | OH | —NHCH(CHOH)COOH |
| 135 | Ti(IV) | OH | NH₂ | —NHCH(CHOH)COOH |
| 136 | Ti(IV) | OH | OH | —NHCH(CH₂COOH)COOH |
| 137 | Ti(IV) | OMe | OH | —NHCH(CH₂COOH)COOH |
| 138 | Ti(IV) | OH | OMe | —NHCH(CH₂COOH)COOH |
| 139 | Ti(IV) | OEt | OH | —NHCH(CH₂COOH)COOH |
| 140 | Ti(IV) | OH | OEt | —NHCH(CH₂COOH)COOH |
| 141 | Ti(IV) | NH₂ | OH | —NHCH(CH₂COOH)COOH |
| 142 | Ti(IV) | OH | NH₂ | —NHCH(CH₂COOH)COOH |
| 143 | Ti(IV) | OH | OH | —NHCH(CH₂CH₂COOH)COOH |
| 144 | Ti(IV) | OMe | OH | —NHCH(CH₂CH₂COOH)COOH |
| 145 | Ti(IV) | OH | OMe | —NHCH(CH₂CH₂COOH)COOH |
| 146 | Ti(IV) | OEt | OH | —NHCH(CH₂CH₂COOH)COOH |
| 147 | Ti(IV) | OH | OEt | —NHCH(CH₂CH₂COOH)COOH |
| 148 | Ti(IV) | NH₂ | OH | —NHCH(CH₂CH₂COOH)COOH |
| 149 | Ti(IV) | OH | NH₂ | —NHCH(CH₂CH₂COOH)COOH |
| 150 | Ti(IV) | OH | OH | —HNCH((CH₂)₄NH₂)COOH |
| 151 | Ti(IV) | OMe | OH | —HNCH((CH₂)₄NH₂)COOH |
| 152 | Ti(IV) | OH | OMe | —HNCH((CH₂)₄NH₂)COOH |
| 153 | Ti(IV) | OEt | OH | —HNCH((CH₂)₄NH₂)COOH |
| 154 | Ti(IV) | OH | OEt | —HNCH((CH₂)₄NH₂)COOH |
| 155 | Ti(IV) | NH₂ | OH | —HNCH((CH₂)₄NH₂)COOH |
| 156 | Ti(IV) | OH | NH₂ | —HNCH((CH₂)₄NH₂)COOH |
| 157 | Ti(IV) | OH | OH | —HNCH((CH₂)₃NH₂)COOH |
| 158 | Ti(IV) | OMe | OH | —HNCH((CH₂)₃NH₂)COOH |
| 159 | Ti(IV) | OH | OMe | —HNCH((CH₂)₃NH₂)COOH |
| 160 | Ti(IV) | OEt | OH | —HNCH((CH₂)₃NH₂)COOH |
| 161 | Ti(IV) | OH | OEt | —HNCH((CH₂)₃NH₂)COOH |
| 162 | Ti(IV) | NH₂ | OH | —HNCH((CH₂)₃NH₂)COOH |
| 163 | Ti(IV) | OH | NH₂ | —HNCH((CH₂)₃NH₂)COOH |
| 164 | Ti(IV) | OH | OH | —HNCH((CH₂)₃NH(CNH₂)═NH)COOH |
| 165 | Ti(IV) | OMe | OH | —HNCH((CH₂)₃NH(CNH₂)═NH)COOH |
| 166 | Ti(IV) | OH | OMe | —HNCH((CH₂)₃NH(CNH₂)═NH)COOH |
| 167 | Ti(IV) | OEt | OH | —HNCH((CH₂)₃NH(CNH₂)═NH)COOH |
| 168 | Ti(IV) | OH | OEt | —HNCH((CH₂)₃NH(CNH₂)═NH)COOH |
| 169 | Ti(IV) | NH₂ | OH | —HNCH((CH₂)₃NH(CNH₂)═NH)COOH |
| 170 | Ti(IV) | OH | NH₂ | —HNCH((CH₂)₃NH(CNH₂)═NH)COOH |
| 171 | Ti(IV) | OH | OH | —HNCH(CH₂CONH₂)COOH |
| 172 | Ti(IV) | OMe | OH | —HNCH(CH₂CONH₂)COOH |
| 173 | Ti(IV) | OH | OMe | —HNCH(CH₂CONH₂)COOH |
| 174 | Ti(IV) | OEt | OH | —HNCH(CH₂CONH₂)COOH |
| 175 | Ti(IV) | OH | OEt | —HNCH(CH₂CONH₂)COOH |
| 176 | Ti(IV) | NH₂ | OH | —HNCH(CH₂CONH₂)COOH |
| 177 | Ti(IV) | OH | NH₂ | —HNCH(CH₂CONH₂)COOH |
| 178 | Ti(IV) | OH | OH | —HNCH(CH₂CH₂CONH₂)COOH |
| 179 | Ti(IV) | OMe | OH | —HNCH(CH₂CH₂CONH₂)COOH |
| 180 | Ti(IV) | OH | OMe | —HNCH(CH₂CH₂CONH₂)COOH |
| 181 | Ti(IV) | OEt | OH | —HNCH(CH₂CH₂CONH₂)COOH |
| 182 | Ti(IV) | OH | OEt | —HNCH(CH₂CH₂CONH₂)COOH |
| 183 | Ti(IV) | NH₂ | OH | —HNCH(CH₂CH₂CONH₂)COOH |
| 184 | Ti(IV) | OH | OH | —(NHCH₂CO)₂OH |
| 185 | Ti(IV) | OH | OH | —(NHCH₂CO)₃OH |
| 186 | Ti(IV) | OH | OH | —(NHCH₂CO)₄OH |
| 187 | Ti(IV) | OH | OH | —(NHCH₂CO)₅OH |
| 188 | Ti(IV) | OH | OH | —(NHCH₂CO)₆OH |
| 189 | Ti(IV) | OH | OH | —(NHCHCH₃CO)₄OH |
| 190 | Ti(IV) | OH | OH | —(NHCH(CHOH)CO)₄OH |
| 191 | Ti(IV) | OH | OH | —(NHCH((CH₂)₃NH₂)CO)₂OH |
| 192 | Ti(IV) | OH | OH | —(NHCH((CH₂)₃NH₂)CO)₃OH |
| 193 | Ti(IV) | OH | OH | —(NHCH((CH₂)₃NH₂)CO)₄OH |
| 194 | Ti(IV) | OH | OH | —(NHCH(CH₂CONH₂)CO)₂OH |
| 195 | Ti(IV) | OH | OH | —N(histidine) |
| 196 | Ti(IV) | OH | OH | —N(proline) |
| 197 | Ti(IV) | OH | OH | —NH(CH₂CH₂O)ₙOH |
| 198 | Ti(IV) | OH | OH | —N(folate) |
| 199 | Ti(IV) | OH | OMe | —NH(CH₂CH₂O)ₙOH |
| 200 | Ti(IV) | OMe | OH | —NH(CH₂CH₂O)ₙOH |

Compounds according to the invention include compounds of formula:

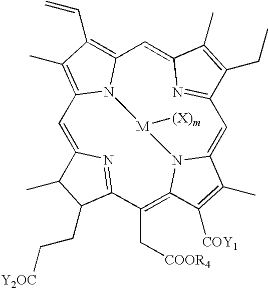

wherein M, Y₁, Y₂, and R₄ are set forth in the table below:

TABLE B

| Compound | | | | |
|---|---|---|---|---|
| No. | M | Y₁ | Y2 | —R₄ |
| 1 | Sn(IV) | OH | OH | —H |
| 2 | Sn(IV) | OMe | OH | —H |
| 3 | Sn(IV) | OH | OMe | —H |
| 4 | Sn(IV) | OEt | OH | —H |
| 5 | Sn(IV) | OH | OEt | —H |
| 6 | Sn(IV) | NH₂ | OH | —H |
| 7 | Sn(IV) | OH | NH₂ | —H |
| 8 | Sn(IV) | OH | OH | —CH₃ |
| 9 | Sn(IV) | OMe | OH | —CH₃ |
| 10 | Sn(IV) | OH | OMe | —CH₃ |
| 11 | Sn(IV) | OEt | OH | —CH₃ |
| 12 | Sn(IV) | OH | OEt | —CH₃ |
| 13 | Sn(IV) | NH₂ | OH | —CH₃ |
| 14 | Sn(IV) | OH | NH₂ | —CH₃ |
| 15 | Sn(IV) | OH | OH | —C₂H₅ |
| 16 | Sn(IV) | OMe | OH | —C₂H₅ |
| 17 | Sn(IV) | OH | OMe | —C₂H₅ |
| 18 | Sn(IV) | OEt | OH | —C₂H₅ |
| 19 | Sn(IV) | OH | OEt | —C₂H₅ |
| 20 | Sn(IV) | NH₂ | OH | —C₂H₅ |
| 21 | Sn(IV) | OH | NH₂ | —C₂H₅ |
| 22 | Sn(IV) | OH | OH | —CH₂Ph |
| 23 | Sn(IV) | OMe | OH | —CH₂Ph |
| 24 | Sn(IV) | OH | OMe | —CH₂Ph |
| 25 | Sn(IV) | OEt | OH | —CH₂Ph |
| 26 | Sn(IV) | OH | OEt | —CH₂Ph |
| 27 | Sn(IV) | NH₂ | OH | —CH₂Ph |
| 28 | Sn(IV) | OH | NH₂ | —CH₂Ph |

In another aspect, the present invention provides a pharmaceutical composition comprising a compound of the invention and a pharmaceutically acceptable carrier.

The compound of the present invention are formulated into final pharmaceutical compositions for administration to the patient or applied to an in vitro target using techniques well-known in the art, for example, as summarized in Remington's Pharmaceutical Sciences, Mack Publishing Co., Easton, Pa. The compositions can be administered systemically, in particular by injection, or can be used topically, sublingually or orally. The compounds of the invention are useful as sonosensitizers, photosensitizers and/or as dual acting sensitizers as therapeutic and diagnostic agents, for example for treatment of several cancer types such as, but not limited to, melanoma, prostate, brain, colon, ovarian, breast, skin, lung, esophagus and bladder cancers and other hormone-sensitive tumors, as well as for treatment of age-related macular degeneration, and for killing cells, viruses, fungi and bacteria in samples and living tissues as well known in the art of PDT and other sonosensitizer applications.

The compounds of the invention are useful, for example, in sensitizing neoplastic cells or other abnormal tissue to destruction by ultrasound frequencies.

The pharmaceutical compositions of the invention will be administered to the patient by standard procedures used in PDT and SDT. The amount of the compound of the invention to be administered to an individual in need and the route of administration will be established according to the experience accumulated with other porphyrins used in SDT, and will vary depending on the choice of the derivative used as active ingredient, the condition, e.g. the kind of tumor, to be treated, the stage of the disease, age and health conditions of the patient, and the judgement of the physician. The preferable routes of administration are sublingual, intravenous or local delivery, such as direct injection into the solid tumor, of a solution or dispersion (e.g., in an aqueous carrier) of the active compound comprising conventional pharmaceutically acceptable carriers and additives, and topical treatment of skin tumors with suitable topical compositions.

The wavelength of the ultrasound is preferably chosen to match the maximum absorbance of the sonosensitizer. The suitable energy for any of the compounds can readily be determined empirically but can be between 20 KHz to 20 MHz, intensity of 0.1 to 500 W/cm2 and duration of 0.5 sec. to 5 hours. In an alternative embodiment, the compounds can be activated by light waves, as typically employed in photodynamic therapy.

The conjugation of proteins, e.g., hormones, growth factors or their derivatives, antibodies, peptides that bind specifically to target cells receptors, and of cell nutrients, e.g. tyrosine, can increase their retention in tumor and treated sites.

The invention further relates to a method of sonodynamic therapy, which comprises administering to an individual in need an effective amount of a compound of the invention, followed by local ultrasound.

The compounds of the invention are also useful for photo- and sonodestruction of normal or malignant animal cells, as desired, as well as of microorganisms in culture, enabling selective photo- and sonodestruction of certain types of cells in culture or infective agents. Thus, the invention further provides the use of the compounds of the invention for in vivo, ex-vivo or in vitro killing of cells or infectious agents such as bacteria, viruses, parasites and fungi in a biological product, e.g. blood, which comprises treating the infected sample with the compound of the invention followed by ultrasound of the sample. Examples of such diseases or conditions include acne, Aids, viral hepatitis, diabetic retinopathy, infection with sars virus, coronary artery stenosis, carotid artery stenosis, intermittent claudication, or Asian (chicken) flu virus, or infections caused by intracellular infectious agents such as Clamidia, tox, ricetzia, rocky mountain spotted fever, q-fever, and others.

The term “cancer” refers to any cancer caused by the proliferation of malignant neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non small cell), breast cancer, pancreatic cancer, melanoma and other skin cancers, stomach cancer, brain tumors, tumors related to Gorlin's syndrome (e.g., medulloblastoma, meningioma, etc.), and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds described herein may be useful in preventing, treating and studying are, for example, colon carcinoma, familiarly adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma. In one aspect of the invention, the present invention provides for the use of one or more compounds of the invention in the manufacture of a medicament for the treatment of cancer.

In one embodiment, the present invention includes the use of one or more compounds of the invention in the manufacture of a medicament that prevents further aberrant proliferation, differentiation, or survival of cells. For example, compounds of the invention may be useful in preventing tumors from increasing in size or from reaching a metastatic state. The subject compounds may be administered to halt or inhibit the progression or advancement of cancer or to induce tumor necrosis, tumor apoptosis, or inhibit tumor angiogenesis.

In addition, the instant invention includes use of the subject compounds to prevent a recurrence of cancer. This is accomplished in part because the use of the compounds in a therapeutic mode creates an inflammatory response that yields a “vaccine effect.”

This invention further embraces the treatment or prevention of cell proliferative disorders such as hyperplasias, dysplasias and pre-cancerous lesions. Dysplasia is the earliest form of pre-cancerous lesion recognizable in a biopsy by a pathologist. The subject compounds may be administered for the purpose of preventing said hyperplasias, dysplasias or pre-cancerous lesions from continuing to expand or from becoming cancerous. Examples of pre-cancerous lesions may occur in skin, esophageal tissue, breast and cervical intra-epithelial tissue.

The compounds of the invention can be further useful in treating inflammatory diseases and autoimmune diseases, including acute and chronic immune and autoimmune pathologies, such as systemic lupus erythematosus (SLE), rheumatoid arthritis, thyroidosis, graft versus host disease, scleroderma, diabetes mellitus, Graves' disease, Beschet's disease, chronic inflammatory pathologies and vascular inflammatory pathologies, including chronic inflammatory pathologies such as sarcoidosis, chronic inflammatory bowel disease, ulcerative colitis, and Crohn's pathology and vascular inflammatory pathologies, such as, but not limited to, disseminated intravascular coagulation, atherosclerosis, and Kawasaki's pathology. Diseases related to angiogenesis or VEGF/VPF, such as ocular neovascularization, psoriasis, duodenal ulcers, angiogenesis of the female reproductive tract can also be treated. As discussed above, infections, such as bacterial, viral or parasitic infections, and related diseases can be treated, including, sepsis syndrome, cachexia, circulatory collapse and shock resulting from acute or chronic bacterial infection, acute and chronic parasitic and/or infectious diseases, bacterial, viral or fungal. Vascular disorders such as atherosclerosis and restenosis result from disregulated growth of the vessel wall, restricting blood flow to vital organs can be treated. Skin diseases, such as acne, psoriasis and inflammatory dermatoses such as dermatitis, eczema, atopic dermatitis, allergic contact dermatitis can also be treated. Additionally, the grey color from hair can be removed. The compounds of the invention can be used alone or in combination with other active agents. Combination therapy includes the administration of the subject compounds in further combination with other biologically active ingredients (such as, but not limited to, a second and different antineoplastic agent) and non-drug therapies (such as, but not limited to, surgery or radiation treatment). For instance, the compounds of the invention can be used in combination with other pharmaceutically active compounds, preferably compounds that are able to enhance the effect of the compounds of the invention. The compounds of the invention can be administered simultaneously (as a single preparation or separate preparation) or sequentially to the other drug therapy. In general, a combination therapy envisions administration of two or more drugs during a single cycle or course of therapy.

Alternatively or additionally, administration of the subject compounds can be staggered, thereby resulting in varied compartmental distribution.

The compounds are administered prior to activation by ultrasound (or phototherapy). Preferably the compounds are administered at least one hour before, generally between 12 and 72 hrs, activation.

In one aspect of the invention, the subject compounds may be administered in combination with one or more separate agents that modulate protein kinases involved in various disease states. Examples of such kinases may include, but are not limited to: serine/threonine specific kinases, receptor tyrosine specific kinases and non-receptor tyrosine specific kinases. Serine/threonine kinases include mitogen activated protein kinases (MAPK), meiosis specific kinase (Aurora), RAF and Aurora kinase. Examples of receptor kinase families include epidermal growth factor receptor (EGFR) (e.g. HER2/neu, HER3, HER4, ErbB, ErbB2, ErbB3, ErbB4, Xmrk, DER, Let23); fibroblast growth factor (FGF) receptor (e.g. FGF-R1, GFF-R2/BEK/CEK3, FGF-R3/CEK2, FGF-R4/TKF, KGF-R); hepatocyte growth/scatter factor receptor (HGFR) (e.g, MET, RON, SEA, SEX); insulin receptor (e.g. IGFI-R); Eph (e.g. CEK5, CEK8, EBK, ECK, EEK, EHK-1, EHK-2, ELK, EPH, ERK, HEK, MDK2, MDK5, SEK); Axl (e.g. Mer/Nyk, Rse); RET; and platelet-derived growth factor receptor (PDGFR) (e.g. PDGFα-R, PDGβ-R, CSF1-R/FMS, SCF-R/C-KIT, VEGF-R/FLT, NEK/FLK1, FLT3/FLK2/STK-1). Non-receptor tyrosine kinase families include, but are not limited to, BCR-ABL (e.g. p43^{abl}, ARG); BTK (e.g. ITK/EMT, TEC); CSK, FAK, FPS, JAK, SRC, BMX, FER, CDK and SYK.

In certain preferred embodiments, the compounds of the invention are administered in combination with a chemotherapeutic agent. Chemotherapeutic agents encompass a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to the cancer or its treatment. Examples of such agents include, but are not limited to, alkylating agents such as mustard gas derivatives (Mechlorethamine, cylophosphamide, chlorambucil, melphalan, ifosfamide), ethylenimines (thiotepa, hexamethylmelanine), Alkylsulfonates (Busulfan), Hydrazines and Triazines (Altretamine, Procarbazine, Dacarbazine and Temozolomide), Nitrosoureas (Carmustine, Lomustine and Streptozocin), Ifosfamide and metal salts (Carboplatin, Cisplatin, and Oxaliplatin); plant alkaloids such as Podophyllotoxins (Etoposide and Tenisopide), Taxanes (Paclitaxel and Docetaxel), Vinca alkaloids (Vincristine, Vinblastine, Vindesine and Vinorelbine), and Camptothecan analogs (Irinotecan and Topotecan); anti-tumor antibiotics such as Chromomycins (Dactinomycin and Plicamycin), Anthracyclines (Doxorubicin, Daunorubicin, Epirubicin, Mitoxantrone, Valrubicin and Idarubicin), and miscellaneous antibiotics such as Mitomycin, Actinomycin and Bleomycin; anti-metabolites such as folic acid antagonists (Methotrexate, Pemetrexed, Raltitrexed, Aminopterin), pyrimidine antagonists (5-Fluorouracil, Floxuridine, Cytarabine, Capecitabine, and Gemcitabine), purine antagonists (6-Mercaptopurine and 6-Thioguanine) and adenosine deaminase inhibitors (Cladribine, Fludarabine, Mercaptopurine, Clofarabine, Thioguanine, Nelarabine and Pentostatin); topoisomerase inhibitors such as topoisomerase I inhibitors (Ironotecan, topotecan) and topoisomerase II inhibitors (Amsacrine, etoposide, etoposide phosphate, teniposide); monoclonal antibodies (Alemtuzumab, Gemtuzumab ozogamicin, Rituximab, Trastuzumab, Ibritumomab Tioxetan, Cetuximab, Panitumumab, Tositumomab, Bevacizumab); and miscellaneous anti-neoplastics such as ribonucleotide reductase inhibitors (Hydroxyurea); adrenocortical steroid inhibitor (Mitotane); enzymes (Asparaginase and Pegaspargase); anti-microtubule agents (Estramustine); and retinoids (Bexarotene, Isotretinoin, Tretinoin (ATRA).

In certain preferred embodiments, the compounds of the invention are administered in combination with a chemoprotective agent. Chemoprotective agents act to protect the body or minimize the side effects of chemotherapy. Examples of such agents include, but are not limited to, amfostine, mesna, and dexrazoxane.

In one aspect of the invention, the subject compounds are administered in combination with radiation therapy. Radiation is commonly delivered internally (implantation of radioactive material near cancer site) or externally from a machine that employs photon (x-ray or gamma-ray) or particle radiation. Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

It will be appreciated that compounds of the invention can be used in combination with an immunotherapeutic agent. One form of immunotherapy is the generation of an active systemic tumor-specific immune response of host origin by administering a vaccine composition at a site distant from the tumor. Various types of vaccines have been proposed, including isolated tumor-antigen vaccines and anti-idiotype vaccines. Another approach is to use tumor cells from the subject to be treated, or a derivative of such cells (reviewed by Schirrmacher et al. (1995) J. Cancer Res. Clin. Oncol. 121:487). In U.S. Pat. No. 5,484,596, Hanna Jr. et al. claims a method for treating a resectable carcinoma to prevent recurrence or metastases, comprising surgically removing the tumor, dispersing the cells with collagenase, irradiating the cells, and vaccinating the patient with at least three consecutive doses of about 10⁷ cells.

It will be appreciated that the compounds of the invention may advantageously be used in conjunction with one or more adjunctive therapeutic agents. Examples of suitable agents for adjunctive therapy include a 5HT₁ agonist, such as a triptan (e.g. sumatriptan or naratriptan); an adenosine A1 agonist; an EP ligand; an NMDA modulator, such as a glycine antagonist; a sodium channel blocker (e.g. lamotrigine); a substance P antagonist (e.g. an NK₁ antagonist); a cannabinoid; acetaminophen or phenacetin; a 5-lipoxygenase inhibitor; a leukotriene receptor antagonist; a DMARD (e.g. methotrexate); gabapentin and related compounds; a tricyclic antidepressant (e.g. amitryptilline); a neurone stabilizing antiepileptic drug; a mono-aminergic uptake inhibitor (e.g. venlafaxine); a matrix metalloproteinase inhibitor; a nitric oxide synthase (NOS) inhibitor, such as an iNOS or an nNOS inhibitor; an inhibitor of the release, or action, of tumour necrosis factor .alpha.; an antibody therapy, such as a monoclonal antibody therapy; an antiviral agent, such as a nucleoside inhibitor (e.g. lamivudine) or an immune system modulator (e.g. interferon); an opioid analgesic; a local anaesthetic; a stimulant, including caffeine; an H₂-antagonist (e.g. ranitidine); a proton pump inhibitor (e.g. omeprazole); an antacid (e.g. aluminum or magnesium hydroxide; an antiflatulent (e.g. simethicone); a decongestant (e.g. phenylephrine, phenylpropanolamine, pseudoephedrine, oxymetazoline, epinephrine, naphazoline, xylometazoline, propylhexedrine, or levo-desoxyephedrine); an antitussive (e.g. codeine, hydrocodone, carmiphen, carbetapentane, or dextramethorphan); a diuretic; or a sedating or non-sedating antihistamine.

In one embodiment, compounds of the invention can be used to induce or inhibit apoptosis, a physiological cell death process critical for normal development and homeostasis. Alterations of apoptotic pathways contribute to the pathogenesis of a variety of human diseases. Compounds of the invention, as modulators of apoptosis, will be useful in the treatment of a variety of human diseases with aberrations in apoptosis including cancer (particularly, but not limited to, follicular lymphomas, carcinomas with p53 mutations, hormone dependent tumors of the breast, prostate and ovary, and precancerous lesions such as familial adenomatous polyposis), viral infections (including, but not limited to, herpes virus, poxvirus, Epstein-Barr virus, Sindbis virus and adenovirus), autoimmune diseases (including, but not limited to, systemic lupus, erythematosus, immune mediated glomerulonephritis, rheumatoid arthritis, psoriasis, inflammatory bowel diseases, and autoimmune diabetes mellitus), neurodegenerative disorders (including, but not limited to, Alzheimer's disease, AIDS-related dementia, Parkinson's disease, amyotrophic lateral sclerosis, retinitis pigmentosa, spinal muscular atrophy and cerebellar degeneration), AIDS, myelodysplastic syndromes, aplastic anemia, ischemic injury associated myocardial infarctions, stroke and reperfusion injury, arrhythmia, atherosclerosis, toxin-induced or alcohol induced liver diseases, hematological diseases (including, but not limited to, chronic anemia and aplastic anemia), degenerative diseases of the musculoskeletal system (including, but not limited to, osteoporosis and arthritis), aspirin-sensitive rhinosinusitis, cystic fibrosis, multiple sclerosis, kidney diseases, and cancer pain.

### DEFINITIONS

Listed below are definitions of various terms used to describe this invention. These definitions apply to the terms as they are used throughout this specification and claims, unless otherwise limited in specific instances, either individually or as part of a larger group.

The term “acyl” refers to hydrogen, alkyl, partially saturated or fully saturated cycloalkyl, partially saturated or fully saturated heterocycle, aryl, and heteroaryl substituted carbonyl groups. For example, acyl includes groups such as (C₁-C₆)alkanoyl (e.g., formyl, acetyl, propionyl, butyryl, valeryl, caproyl, t-butylacetyl, etc.), (C₃-C₆)cycloalkylcarbonyl (e.g., cyclopropylcarbonyl, cyclobutylcarbonyl, cyclopentylcarbonyl, cyclohexylcarbonyl, etc.), heterocyclic carbonyl (e.g., pyrrolidinylcarbonyl, pyrrolid-2-one-5-carbonyl, piperidinylcarbonyl, piperazinylcarbonyl, tetrahydrofuranylcarbonyl, etc.), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, benzo[b]thiophenyl-2-carbonyl, etc.). In addition, the alkyl, cycloalkyl, heterocycle, aryl and heteroaryl portion of the acyl group may be any one of the groups described in the respective definitions. When indicated as being “optionally substituted”, the acyl group may be unsubstituted or optionally substituted with one or more substituents (typically, one to three substituents) independently selected from the group of substituents listed below in the definition for “substituted” or the alkyl, cycloalkyl, heterocycle, aryl and heteroaryl portion of the acyl group may be substituted as described above in the preferred and more preferred list of substituents, respectively.

The term “alkyl” embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about eight carbon atoms. Examples of such radicals include methyl, ethyl, n-propyl, isopropyl, n-butyl, isobutyl, sec-butyl, tert-butyl, pentyl, iso-amyl, hexyl and the like.

The term “alkenyl” embraces linear or branched radicals having at least one carbon-carbon double bond of two to about twenty carbon atoms or, preferably, two to about twelve carbon atoms. More preferred alkenyl radicals are “lower alkenyl” radicals having two to about ten carbon atoms and more preferably about two to about eight carbon atoms. Examples of alkenyl radicals include ethenyl, allyl, propenyl, butenyl and 4-methylbutenyl. The terms “alkenyl”, and “lower alkenyl”, embrace radicals having “cis” and “trans” orientations, or alternatively, “E” and “Z” orientations.

The term “alkynyl” embraces linear or branched radicals having at least one carbon-carbon triple bond of two to about twenty carbon atoms or, preferably, two to about twelve carbon atoms. More preferred alkynyl radicals are “lower alkynyl” radicals having two to about ten carbon atoms and more preferably about two to about eight carbon atoms. Examples of alkynyl radicals include propargyl, 1-propynyl, 2-propynyl, 1-butyne, 2-butynyl and 1-pentynyl.

The term “cycloalkyl” embraces saturated carbocyclic radicals having three to about twelve carbon atoms. The term “cycloalkyl” embraces saturated carbocyclic radicals having three to about twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The term “cycloalkenyl” embraces partially unsaturated carbocyclic radicals having three to twelve carbon atoms. Cycloalkenyl radicals that are partially unsaturated carbocyclic radicals that contain two double bonds (that may or may not be conjugated) can be called “cycloalkyldienyl”. More preferred cycloalkenyl radicals are “lower cycloalkenyl” radicals having four to about eight carbon atoms. Examples of such radicals include cyclobutenyl, cyclopentenyl and cyclohexenyl.

The term “alkoxy” or “alkoxide” embraces linear or branched oxy-containing radicals each having alkyl portions of one to about twenty carbon atoms or, preferably, one to about twelve carbon atoms. More preferred alkoxy radicals are “lower alkoxy” radicals having one to about ten carbon atoms and more preferably having one to about eight carbon atoms. Examples of such radicals include methoxy, ethoxy, propoxy, butoxy and tert-butoxy.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl.

The terms “heterocyclyl”, “heterocycle” “heterocyclic” or “heterocyclo” embrace saturated, partially unsaturated and unsaturated heteroatom-containing ring-shaped radicals, which can also be called “heterocyclyl”, “heterocycloalkenyl” and “heteroaryl” correspondingly, where the heteroatoms may be selected from nitrogen, sulfur and oxygen. Examples of saturated heterocyclyl radicals include saturated 3 to 6-membered heteromonocyclic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole. Heterocyclyl radicals may include a pentavalent nitrogen, such as in tetrazolium and pyridinium radicals. The term “heterocycle” also embraces radicals where heterocyclyl radicals are fused with aryl or cycloalkyl radicals. Examples of such fused bicyclic radicals include benzofuran, benzothiophene, and the like.

The term “heteroaryl” embraces unsaturated heterocyclyl radicals. Examples of heteroaryl radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like.

The term “alkylthio” embraces radicals containing a linear or branched alkyl radical, of one to about ten carbon atoms attached to a divalent sulfur atom. Preferred alkylthio radicals have alkyl radicals of one to about twenty carbon atoms or, preferably, one to about twelve carbon atoms. More preferred alkylthio radicals have alkyl radicals are “lower alkylthio” radicals having one to about ten carbon atoms. Most preferred are alkylthio radicals having lower alkyl radicals of one to about eight carbon atoms. Examples of such lower alkylthio radicals are methylthio, ethylthio, propylthio, butylthio and hexylthio.

The term “aminoalkyl” embraces alkyl radicals substituted with amino radicals. Preferred aminoalkyl radicals have alkyl radicals having about one to about twenty carbon atoms or, preferably, one to about twelve carbon atoms. More preferred aminoalkyl radicals are “lower aminoalkyl” that have alkyl radicals having one to about ten carbon atoms. Most preferred are aminoalkyl radicals having lower alkyl radicals having one to eight carbon atoms. Examples of such radicals include aminomethyl, aminoethyl, and the like.

The term “substituted” refers to the replacement of one or more hydrogen radicals in a given structure with the radical of a specified substituent including, but not limited to: halo, alkyl, alkenyl, alkynyl, aryl, heterocyclyl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, aminocarbonylcycloalkyl, aminocarbonylheterocyclyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and aliphatic. It is understood that the substituent may be further substituted.

For simplicity, chemical moieties are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g. CH₃—CH₂—), or in other instances, a bivalent linking moiety can be “alkyl,” in which case those skilled in the art will understand the alkyl to be a divalent radical (e.g., —CH₂—CH₂—), which is equivalent to the term “alkylene.”

The terms “halogen” or “halo” as used herein, refers to an atom selected from fluorine, chlorine, bromine and iodine.

The term “activated therapy”, as used herein, refers to photodynamic therapy, sonodynamic therapy or a combination thereof.

The phrase “adjunctive therapy” encompasses treatment of a subject with agents that reduce or avoid side effects associated with the combination therapy of the present invention, including, but not limited to, those agents, for example, that reduce the toxic effect of anticancer drugs, e.g., bone resorption inhibitors, cardioprotective agents; prevent or reduce the incidence of nausea and vomiting associated with chemotherapy, radiotherapy or operation; or reduce the incidence of infection associated with the administration of myelosuppressive anticancer drugs.

The term “angiogenesis,” as used herein, refers to the formation of blood vessels. Specifically, angiogenesis is a multi-step process in which endothelial cells focally degrade and invade through their own basement membrane, migrate through interstitial stroma toward an angiogenic stimulus, proliferate proximal to the migrating tip, organize into blood vessels, and reattach to newly synthesized basement membrane (see Folkman et al., Adv. Cancer Res., Vol. 43, pp. 175-203 (1985)). Anti-angiogenic agents interfere with this process. Examples of agents that interfere with several of these steps include thrombospondin-1, angiostatin, endostatin, interferon alpha, and compounds such as matrix metalloproteinase (MMP) inhibitors that block the actions of enzymes that clear and create paths for newly forming blood vessels to follow; compounds, such as .alpha.v.beta.3 inhibitors, that interfere with molecules that blood vessel cells use to bridge between a parent blood vessel and a tumor; agents, such as specific COX-2 inhibitors, that prevent the growth of cells that form new blood vessels; and protein-based compounds that simultaneously interfere with several of these targets.

The term “apoptosis” as used herein refers to programmed cell death as signaled by the nuclei in normally functioning human and animal cells when age or state of cell health and condition dictates. An “apoptosis inducing agent” triggers the process of programmed cell death.

The term “cancer” as used herein denotes a class of diseases or disorders characterized by uncontrolled division of cells and the ability of these cells to invade other tissues, either by direct growth into adjacent tissue through invasion or by implantation into distant sites by metastasis.

The term “compound” is defined herein to include pharmaceutically acceptable salts, solvates, hydrates, polymorphs, enantiomers, diastereoisomers, racemates and the like of the compounds having a formula as set forth herein.

As used herein, the term “dysplasia” refers to abnormal cell growth, and typically refers to the earliest form of pre-cancerous lesion recognizable in a biopsy by a pathologist.

As used herein, the term “effective amount of the subject compounds,” with respect to the subject method of treatment, refers to an amount of the subject compound which, when delivered as part of desired dose regimen, brings about, e.g. a change in the rate of cell proliferation and/or state of differentiation and/or rate of survival of a cell to clinically acceptable standards. This amount may further relieve to some extent one or more of the symptoms of a neoplasia disorder, including, but is not limited to: 1) reduction in the number of cancer cells; 2) reduction in tumor size; 3) inhibition (i.e., slowing to some extent, preferably stopping) of cancer cell infiltration into peripheral organs; 4) inhibition (i.e., slowing to some extent, preferably stopping) of tumor metastasis; 5) inhibition, to some extent, of tumor growth; 6) relieving or reducing to some extent one or more of the symptoms associated with the disorder; and/or 7) relieving or reducing the side effects associated with the administration of anticancer agents.

The term “hyperplasia,” as used herein, refers to excessive cell division or growth.

The phrase an “immunotherapeutic agent” refers to agents used to transfer the immunity of an immune donor, e.g., another person or an animal, to a host by inoculation. The term embraces the use of serum or gamma globulin containing performed antibodies produced by another individual or an animal; nonspecific systemic stimulation; adjuvants; active specific immunotherapy; and adoptive immunotherapy. Adoptive immunotherapy refers to the treatment of a disease by therapy or agents that include host inoculation of sensitized lymphocytes, transfer factor, immune RNA, or antibodies in serum or gamma globulin.

The term “inhibition,” in the context of neoplasia, tumor growth or tumor cell growth, may be assessed by delayed appearance of primary or secondary tumors, slowed development of primary or secondary tumors, decreased occurrence of primary or secondary tumors, slowed or decreased severity of secondary effects of disease, arrested tumor growth and regression of tumors, among others. In the extreme, complete inhibition, is referred to herein as prevention or chemoprevention.

The term “metastasis,” as used herein, refers to the migration of cancer cells from the original tumor site through the blood and lymph vessels to produce cancers in other tissues. Metastasis also is the term used for a secondary cancer growing at a distant site.

The term “necrosis”, as used herein, refers to death of cells or tissues through injury or disease, especially in a localized area of the body.

The term “neoplasm,” as used herein, refers to an abnormal mass of tissue that results from excessive cell division. Neoplasms may be benign (not cancerous), or malignant (cancerous) and may also be called a tumor. The term “neoplasia” is the pathological process that results in tumor formation.

As used herein, the term “pre-cancerous” refers to a condition that is not malignant, but is likely to become malignant if left untreated.

The term “proliferation” refers to cells undergoing mitosis.

The phrase a “radio therapeutic agent” refers to the use of electromagnetic or particulate radiation in the treatment of neoplasia.

The term “recurrence” as used herein refers to the return of cancer after a period of remission. This may be due to incomplete removal of cells from the initial cancer and may occur locally (the same site of initial cancer), regionally (in vicinity of initial cancer, possibly in the lymph nodes or tissue), and/or distally as a result of metastasis.

The term “treatment” refers to any process, action, application, therapy, or the like, wherein a mammal, including a human being, is subject to medical aid with the object of improving the mammal's condition, directly or indirectly.

The term “vaccine” includes agents that induce the patient's immune system to mount an immune response against the tumor by attacking cells that express tumor associated antigens (Teas).

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid or inorganic acid. Examples of pharmaceutically acceptable nontoxic acid addition salts include, but are not limited to, salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid, oxalic acid, lactic acid, lactobionic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound which is convertible in vivo by metabolic means (e.g. by hydrolysis) to a compound of the invention. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development”, Chapter 5, 113-191 (1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

As used herein, “pharmaceutically acceptable carrier” is intended to include any and all solvents, dispersion media, coatings, antibacterial and antifungal agents, isotonic and absorption delaying agents, and the like, compatible with pharmaceutical administration, such as sterile pyrogen-free water. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

As used herein, the term “pre-cancerous” refers to a condition that is not malignant, but is likely to become malignant if left untreated.

The term “subject” as used herein refers to an animal. Preferably the animal is a mammal. More preferably the mammal is a human. A subject also refers to, for example, dogs, cats, horses, cows, pigs, guinea pigs, fish, birds and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers and/or cis- and trans-isomers. Likewise, all tautomeric forms are also intended to be included. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

### Pharmaceutical Compositions

The pharmaceutical compositions of the present invention comprise a therapeutically effective amount of a compound of the present invention formulated together with one or more pharmaceutically acceptable carriers or excipients. The compound and/or composition can be sterile and suitable for injection.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; cyclodextrins such as alpha- (α), beta- (β) and gamma- (γ) cyclodextrins; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered sublingually, orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound can be mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to VanDevanter et al., U.S. Pat. No. 5,508,269 to Smith et al., and WO 98/43650 by Montgomery, all of which are incorporated herein by reference).

By a “therapeutically effective amount” of a compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the compound described above may range from about 0.01 mg/Kg to about 500 mg/Kg, preferably from about 0.1 to about 10 mg/Kg. Effective doses will also vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 10 mg/kg body weight. Single dose compositions may contain such amounts or submultiples thereof to make up the daily dose. In general, treatment regimens according to the present invention comprise administration to a patient in need of such treatment from about 10 mg to about 1000 (200) mg of the compound(s) of this invention per day in single or multiple doses.

The compounds of the formulae described herein can, for example, be administered by injection, intravenously, intraarterially, subdermally, intraperitoneally, intramuscularly, or subcutaneously; or orally, sublingually, buccally, nasally, transmucosally, topically, in an ophthalmic preparation, or by inhalation, with a dosage ranging from about 0.01 to about 500 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with pharmaceutically excipients or carriers to produce a single dosage form will vary depending upon the host treated and the particular mode of administration. A typical preparation will contain from about 5% to about 100% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

### EXAMPLES

The compounds of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims. Compounds of the invention can be prepared by procedures known to those skilled in the art, such as U.S. Pat. No. 6,462,192 which is hereby incorporated by reference by its entirety.

### Example 1

### Compound SF2

### 1) LC₅₀ Determination:

Since antiangiogenic activity will be assessed at 48 hours post-fertilization (hpf) after treating 20 hpf zebrafish for 28 hours, we determined the LC₅₀ of SF2 using the same treatment regimen. 20 hpf zebrafish (n=30) were treated with SF2 at: 100, 200, 300, 400, 500, 600, 750, 850, 1000, 1500 and 2000 μM for 28 hours at 28° C. and lethality was recorded at 48 hpf. Significant lethality was not observed. No lethality was observed in the repeat experiment. No further testing was performed.

### 2) Assessment of Cytotoxicity for Melanoma Cancer Cell Line WM-266-4:

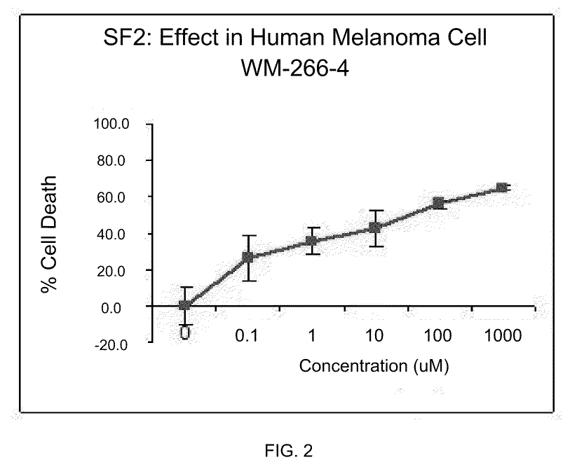

SF2 exhibited significant cytotoxic effect on human melanoma cancer cells WM-266-4 in vitro. A dose response effect was observed (FIG. 2); 35.5, 42.7, 56.0, and 64.8% cell death was observed at: 1, 10, 100 and 1000 μM concentration, respectively.

### Materials and Methods

Compounds: SF2 was pre-weighed. 20 mM stock solution in fish water was prepared. The stock solution was stored at 4° C. before use. Fish water was used as carrier control.

Standard procedures for embryo collection: Embryos were generated by natural pair-wise mating, as described in the Zebrafish Handbook (Westerfield 1993). Four to 5 pairs of adult zebrafish were set up for each mating, and, on average, 100-150 embryos per pair were generated. Embryos were maintained at 28° C. in fish water [200 mg Instant Ocean Salt (Aquarium Systems, Mentor, Ohio) per liter of deionized water; pH 6.6-7.0 maintained with 2.5 mg/liter Jungle pH Stabilizer (Jungle Laboratories Corporation, Cibolo, Tex.); conductivity 670-760 μS]. Embryos were cleaned (dead embryos removed) and sorted by developmental stage (Kimmel, Ballard et al. 1995) at 6 and 24 hpf. Because the embryo receives nourishment from an attached yolk sac, no feeding was required for seven days post fertilization (dpf).

Compound treatment for LC₅₀ determination: 20 hpf zebrafish were distributed into 6-well culture plates, thirty zebrafish per well in 100 μl fish water. SF2 was added to the fish water and zebrafish were exposed by semi-static immersion for 28 hours. Untreated zebrafish were used as control. Repeat sets of experiment were performed; we used SF2 at: 100, 200, 300, 400, 500, 600, 750, 850, 1000, 1500, and 2000 μM.

Lethality curves: Mortality was recorded at 48 hpf. Best-fit concentration-response curves were generated using MS EXCEL. Since the highest mortality observed was 5%, LC₅₀ was not calculated.

Melanoma cell culture conditions: Melanoma cell line WM-266-4, derived from a metastatic site of a malignant melanoma, was cultured in Eagles Minimum Essential Medium (EMEM, Invitrogen, Carlsbad, Calif.) at 37° C., supplemented with 10% fetal bovine serum (Hyclone, Logan, Utah) and grown to approximately 80% confluence before harvesting for cytotoxicity study.

Drug treatment: Two units per ml of dispase (Gibco) was added to the WM-266-4 culture flask and incubated at 37° C. for 30 minutes to achieve a single cell suspension, which was centrifuged at 2500 rpm for 5 min to recover a packed cell pellet, the pellet was re-suspended in the culture media to obtain ˜5×10⁶ cells/ml density. 100 μl (˜5×10⁵ cells) of the cell suspension was distributed to the wells of 96-well microplate; 10 μl of carrier or SF2 solution was added to each well, and incubation was performed at 37° C. for 24 hours. We used 5 concentrations, one log apart: 0.1, 1, 10, 100 and 1000 μM to determine the concentration range that can cause cell death. Six wells were used for each concentration. The number of cells was determined by CyQuant cell proliferation assay (see below).

*Quantitation of melanoma cells using CyQuant proliferation assay: To quantify the total number of melanoma cells, we used CyQUANT™ dye, a well developed reagent for quantifying cell numbers in cell culture (Molecular Probes). After drug treatment, we centrifuged the 96-well culture plate to precipitate melanoma cells; the supernatant was carefully aspirated off; cells were lysed by freezing and thawing. After thawing, 200 μl of CyQUANT™ reagent solution was then added to each well. The dye reacted with total nucleic acid to produce a soluble fluorescent end product, which was measured at 480/520 nm (excitation/emission) within 2-5 minutes using a microplate reader. A standard curve with known number of cells was set up for each experiment; the number of melanoma cells in the sample was calculated against standards. Since the linearity of the standard curve did not extend to 5×10⁶ cells, we used mean relative fluorescence unit (RFU) to calculate the drug effect following formula (b):

% cell death=(1−(RFU(drug treated)/RFU(vehicle control)))×100% (b)

A dose response curve was generated by plotting % cell death vs. concentration; LC₅₀ for melanoma cytotoxicity was calculated based on the dose response curve.

### Results

1) Determination of LC₅₀:

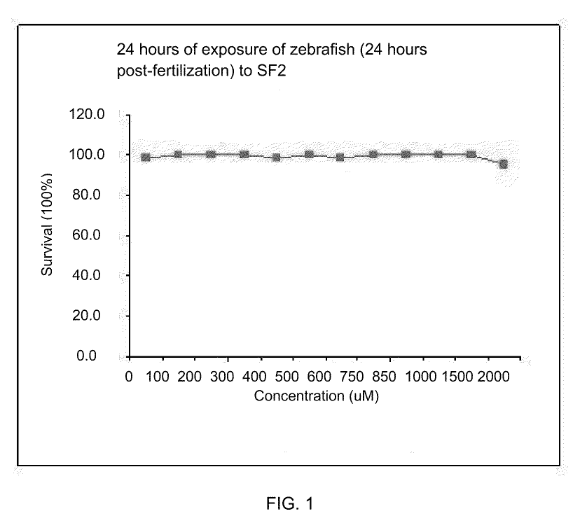

LC₅₀ determination was estimated in repeated experiments: 20 hpf zebrafish were treated with SF2 for 28 hours at 28° C. at: 100, 200, 300, 400, 500, 600, 750, 850, 1000, 1500 and 2000 μM. Significant lethality was not observed in either experiment (Table 1). The concentration-response curves were generated as shown in FIG. 1. The highest lethality observed for SF2 at the highest soluble concentration (2000 μM) was 5.0%. 50% lethality was not reached; LC₅₀ was not determined.

TABLE I
Results of LC50 determination.

| | # Dead | | # Dead | | | |
|---|---|---|---|---|---|---|
| Concen- | Fish | | Fish | | Mean | |
| tration | (exp | Mortality | (exp | Mortality | Mortality | Survival |
| (uM) | 1) | (%) | 2) | (%) | (%) | (%) |
| 0 | 1 | 3.3 | 0 | 0.0 | 1.7 | 98.3 |
| 100 | 0 | 0.0 | 0 | 0.0 | 0.0 | 100.0 |
| 200 | 0 | 0.0 | 0 | 0.0 | 0.0 | 100.0 |
| 300 | 0 | 0.0 | 0 | 0.0 | 0.0 | 100.0 |
| 400 | 1 | 3.3 | 0 | 0.0 | 1.7 | 98.3 |
| 500 | 0 | 0.0 | 0 | 0.0 | 0.0 | 100.0 |
| 600 | 1 | 3.3 | 0 | 0.0 | 1.7 | 98.3 |
| 750 | 0 | 0.0 | 0 | 0.0 | 0.0 | 100.0 |
| 850 | 0 | 0.0 | 0 | 0.0 | 0.0 | 100.0 |
| 1000 | 0 | 0.0 | 0 | 0.0 | 0.0 | 100.0 |
| 1500 | 0 | 0.0 | 0 | 0.0 | 0.0 | 100.0 |
| 2000 | 3 | 10.0 | 0 | 0.0 | 5.0 | 95.0 |

FIG. 1 showed no lethality at the highest concentration. Since lethality was not observed at the highest concentration. The curve appeared as a flat line.

2). Assessment of Cytotoxicity for Melanoma Cancer Cell WM-266-4.

SF2 cytotoxicity for melanoma cancer cell WM-266-4 was assessed at 0.1, 1, 10, 100, and 1000 μM, 6 wells were used for each concentration, mean fluorescence was used to calculate drug effect; results are shown in Tables II and III. SF2 caused very significant (P=0.0001 using one way ANOVA) death of human melanoma cancer cell line WM-266-4; Dunnett's test identified significant effect at: 1, 10, 100 and 1000 μM concentrations (Table II), which caused 35.5, 42.7, 56.0, and 64.8%, respectively, death in human melanoma cancer cell WM-266-4 (Table III).

TABLE II
One-way analysis of variance (One-way ANOVA)

| | |
|---|---|
| P value | 0.0001 |
| Are means signif. different? (P < 0.05) | Yes |
| Number of groups | 6 |

| Dunnett's Multiple | Mean | | | |
|---|---|---|---|---|
| Comparison Test | Diff. | q | P value | 95% CI of diff |
| 0 vs 0.1 | 7849 | 2.185 | P > 0.05 | −1706 to 17400 |
| 0 vs 1 | 10570 | 2.942 | P < 0.05 | 1015 to 20120 |
| 0 vs 10 | 12740 | 3.547 | P < 0.01 | 3186 to 22300 |
| 0 vs 100 | 16680 | 4.644 | P < 0.01 | 7126 to 26240 |
| 0 vs 1000 | 19300 | 5.373 | P < 0.01 | 9745 to 28850 |

TABLE III
Results of cytotoxicity assessment for human melanoma cancer cell
WM-266-4

| | | | | | SD | | |
|---|---|---|---|---|---|---|---|
| SF2 | | | % of | % of Cell | % of | | SE %/of |
| (uM) | Mean | SD | Control | Death | Control | SE | Control |
| 0 | 29806 | 7194 | 100.0 | 0.0 | 24.1 | 2936 | 9.9 |
| 0.1 | 21958 | 9590 | 73.7 | 26.3 | 32.2 | 3914 | 13.1 |
| 1 | 19237 | 5279 | 64.5 | 35.5 | 17.7 | 2155 | 7.2 |
| 10 | 17066 | 7482 | 57.3 | 42.7 | 25.1 | 3054 | 10.2 |
| 100 | 13125 | 1938 | 44.0 | 56.0 | 6.5 | 791 | 2.7 |
| 1000 | 10506 | 956 | 35.2 | 64.8 | 3.2 | 390 | 1.3 |

Based on the results, a dose-response curve was generated as shown in FIG. 2. Compound SF2 was found not toxic to zebrafish; however, it was very toxic to human melanoma cancer cell WM-266-4.

### Example 2

### Compound SF1

### 1) LC₅₀ Determination:

Since antiangiogenic activity will be assessed at 48 hours post-fertilization (hpf) after treating 20 hpf zebrafish for 28 hours, we determined the LC₅₀ of SF1 using the same treatment regimen. 20 hpf zebrafish (n=30) were treated with SF1 at: 1, 10, 100, 1000 and 2000 μM for 28 hours at 28° C. and lethality was recorded at 48 hpf. No lethality was observed up to 2000 μM. No further testing was performed.

### 2) Assessment of Cytotoxicity for Melanoma Cancer Cell Line WM-266-4:

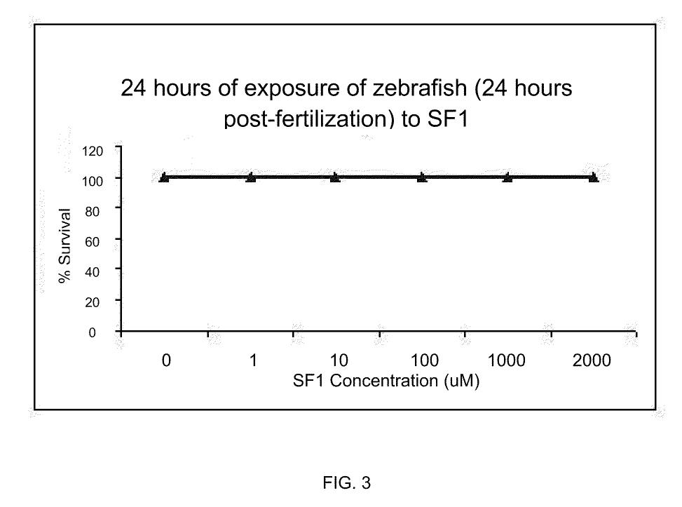

SF1 exhibited significant cytotoxic effect on human melanoma cancer cells WM-266-4 in vitro. A dose response effect was observed (FIG. 3); 57%, 81%, and 87% cell death was observed at: 100, 1000 and 2000 μM concentration, respectively.

### Materials and Methods

Compounds: SF1 was pre-weighed. 100 mM stock solution in fish water was prepared. The stock solution was stored at 4° C. before use. Fish water was used as carrier control.

Standard procedures for embryo collection: Embryos were generated by natural pair-wise mating, as described in the Zebrafish Handbook (Westerfield 1993). Four to 5 pairs of adult zebrafish were set up for each mating, and, on average, 100-150 embryos per pair were generated. Embryos were maintained at 28° C. in fish water [200 mg Instant Ocean Salt (Aquarium Systems, Mentor, Ohio) per liter of deionized water; pH 6.6-7.0 maintained with 2.5 mg/liter Jungle pH Stabilizer (Jungle Laboratories Corporation, Cibolo, Tex.); conductivity 670-760 μS]. Embryos were cleaned (dead embryos removed) and sorted by developmental stage (Kimmel, Ballard et al. 1995) at 6 and 20 hpf. Because the embryo receives nourishment from an attached yolk sac, no feeding was required for seven days post fertilization (dpf).

Compound treatment for LC₅₀ determination: 20 hpf zebrafish were distributed into 6-well culture plates, thirty zebrafish per well in 100 μl fish water. SF1 was added to the fish water and zebrafish were exposed by semi-static immersion for 28 hours. Untreated zebrafish were used as control. Repeat sets of experiment were performed; we used SF1 at: 1, 10, 100, 1000, and 2000 μM.

Lethality curves: Mortality was recorded at 48 hpf. Best-fit concentration-response curves were generated using MS EXCEL. Since mortality was not observed up to 2000 μM, LC₅₀ was not calculated.

Melanoma cell culture conditions: Melanoma cell line WM-266-4, derived from a metastatic site of a malignant melanoma, was cultured in Eagles Minimum Essential Medium (EMEM, Invitrogen, Carlsbad, Calif.) at 37° C., supplemented with 10% fetal bovine serum (Hyclone, Logan, Utah) and grown to approximately 80% confluence before harvesting for cytotoxicity study.

Drug treatment: Two units per ml of dispase (Gibco) was added to the WM-266-4 culture flask and incubated at 37° C. for 30 minutes to achieve a single cell suspension, which was centrifuged at 2500 rpm for 5 min to recover a packed cell pellet, the pellet was re-suspended in the culture media to obtain ˜5×10⁶ cells/ml density. 100 μl (˜5×10⁵ cells) of the cell suspension was distributed to the wells of 96-well microplate; SF1 solution was added to each well, and incubation was performed at 37° C. for 24 hours. We used 5 concentrations: 1, 10, 100, 1000, and 2000 μM to determine the concentration range that can cause cell death. Six wells were used for each concentration. The number of cells was determined by CyQuant cell proliferation assay (see below).

*Quantitation of melanoma cells using CyQuant proliferation assay: To quantify the total number of melanoma cells, we used CyQUANT™ dye, a well developed reagent for quantifying cell numbers in cell culture (Molecular Probes). After drug treatment, we centrifuged the 96-well culture plate to precipitate melanoma cells; the supernatant was carefully aspirated off; cells were lysed by freezing and thawing. After thawing, 200 μl of CyQUANT™ reagent solution was then added to each well. The dye reacted with total nucleic acid to produce a soluble fluorescent end product, which was measured at 480/520 nm (excitation/emission) within 2-5 minutes using a microplate reader. Standard curves with known number of cells were set up for each experiment; based on the standard curve, the number of melanoma cells in each condition was calculated, the mean number of cells in each well was used to calculate the drug effect following formula (b):

% cell death=(1−(Cell number (drug treated)/Cell number (vehicle control)))×100% (b)

A dose response curve was generated by plotting % cell death vs. concentration.

### Results

1) Determination of LC₅₀:

LC₅₀ determination was estimated in repeated experiments: 20 hpf zebrafish were treated with SF1 for 28 hours at 28° C. at: 1, 10, 100, 1000, and 2000 μM. Lethality was not observed (Table IV). The concentration-response curves were generated as shown in FIG. 3. The highest lethality observed for SF1 at the highest soluble concentration (2000 μM) was 5.0%. 50% lethality was not reached; LC₅₀ was not determined.

TABLE IV
Results of LC50 determination.

| | # Dead | | # Dead | | | |
|---|---|---|---|---|---|---|
| Concen- | Fish | | Fish | | Mean | |
| tration | (exp | Mortality | (exp | Mortality | Mortality | Survival |
| (uM) | 1) | (%) | 2) | (%) | (%) | (%) |
| 0 | 0 | 3.3 | 0 | 0.0 | 0.0 | 100.0 |
| 1 | 0 | 0.0 | 0 | 0.0 | 0.0 | 100.0 |
| 10 | 0 | 0.0 | 0 | 0.0 | 0.0 | 100.0 |
| 100 | 0 | 0.0 | 0 | 0.0 | 0.0 | 100.0 |
| 1000 | 0 | 0.0 | 0 | 0.0 | 0.0 | 100.0 |
| 2000 | 0 | 0.0 | 0 | 0.0 | 0.0 | 100.0 |

2). Assessment of Cytotoxicity for Melanoma Cancer Cell WM-266-4.

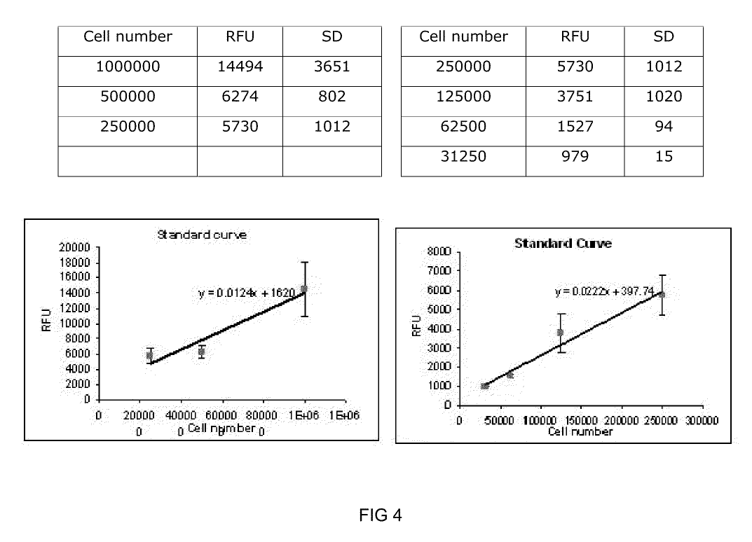

SF1 cytotoxicity for melanoma cancer cell WM-266-4 was assessed at 1, 10, 100, 1000, and 2000 μM; 6 wells were used for each concentration; mean cell number for each group was calculated based on the standard curves (FIG. 4) and was used to calculate drug effect; results are shown in Tables II and III. SF1 caused very significant (P<0.0001 using one way ANOVA) death of human melanoma cancer cell line WM-266-4; Dunnett's test identified significant effect at 100, 1000, and 2000 μM concentrations (Table V), which caused 57, 81, and 87%, respectively, death in human melanoma cancer cell WM-266-4 (Table VI).

TABLE V
One-way analysis of variance (One-way ANOVA)

| | |
|---|---|
| P value | <0.0001 |
| Are means signif. different? (P < 0.05) | Yes |
| Number of groups | 6 |

| Dunnett's Multiple | Mean | | | |
|---|---|---|---|---|
| Comparison Test | Diff. | q | P value | 95% CI of diff |
| 0 vs 1 | 1641 | 2.565 | P > 0.05 | −60.52 to 3343 |
| 0 vs 10 | 69.17 | 0.1081 | P > 0.05 | −1633 to 1771 |
| 0 vs 100 | 2330 | 3.642 | P < 0.01 | 628.0 to 4032 |
| 0 vs 1000 | 4119 | 6.438 | P < 0.01 | 2417 to 5821 |
| 0 vs 2000 | 4631 | 7.238 | P < 0.01 | 2929 to 6333 |

TABLE VI
Results of cytotoxicity assessment for human melanoma
cancer cell WM-266-4

| | Mean | SD | % of cell | | |
|---|---|---|---|---|---|
| SF1 (uM) | (cell #) | (Cell #) | death | SD (%) | SE (%) |
| 0 | 344034 | 79063 | 0 | 23 | 9.4 |
| 1 | 359326 | 111434 | −4 | 32 | 13.2 |
| 10 | 335549 | 78258 | 2 | 23 | 9.3 |
| 100 | 147444 | 24512 | 57 | 7 | 2.9 |
| 1000 | 66851 | 9354 | 81 | 3 | 1.1 |
| 2000 | 43788 | 10132 | 87 | 3 | 1.2 |

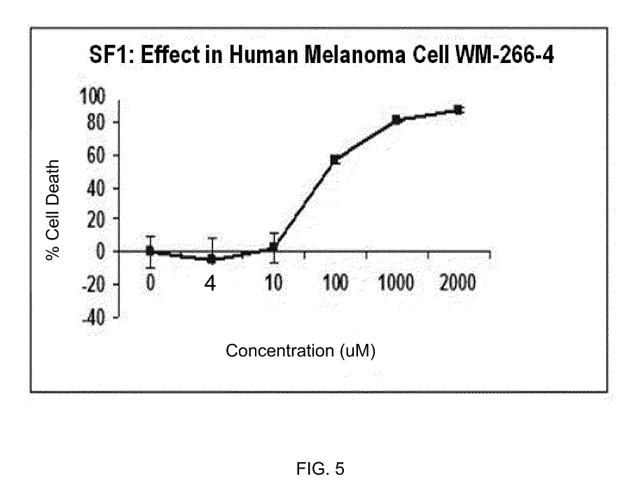

Based on the results, a dose-response curve was generated as shown in FIG. 5.

Compound SF1 was found not toxic to zebrafish; however, it was very toxic to human melanoma cancer cell line WM-266-4.

### Example 3

### Compound SF1

In this study the effect of SDT with SF1 on S-180 sarcoma in mice was examined. The tumor bearing mice allocated to following groups 1) sham-treatment (control, C); 2) ultrasound treatment (only ultrasound treatment, 1.2 W/cm², without SF1 U); 3) SF1 treatment (SF1 20 mg/Kg intraperitoneal, ip) without ultrasound treatment, S); 4) SF1+ultrasound treatment (SU). Following treatment, tumor volume was monitored. The tumor growth inhibition was seen only in group SU, and with increasing ultrasound intensity, the inhibitive effect was enhanced. The tumor growth inhibition was also visible even when covered by barrier of bone. Pathological slices showed coagulated necrosis or metamorphic tissue with inflammatory reaction in the tumor taken from 2 hrs to 36 hrs after SDT. These data revealed that SDT with SF1 inhibited growth of mouse S-180 sarcoma and the inhibitive effect was sound intensity dependant. SDT also induced some inflammation while it destroyed the tumor indicative of a “vaccine” affect. SF1 shows great promise for clinical use in the future.

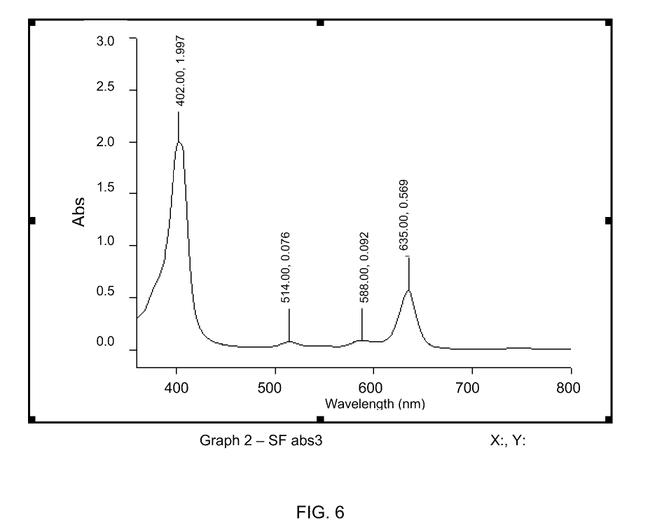

SF1 has an average molecular weight of 942. The sensitizer has photosensitizer capabilities. SF1 absorption spectrum is shown in FIG. 6. SF1 has two absorption peaks. They occur at wavelength 402 nm and 636 nm.

The agent was dissolved in 0.1 mol PSA under sterile conditions in dark room. Its final concentration was adjusted to 2 mg/ml. The container of the suspension was constantly protected from any exposure to room and sunlight, placed into a thermos bottle to shield from light and sound, and stored in refrigerator at 4° C. to 16° C.

The animal tumor model used in this study was KM mouse S-180 sarcoma. The mouse S-180 sarcoma cell line was injected into and raised in the abdominal cavity of the KM mouse, and regenerated three times by passing malignant ascites from one mouse to another. Then the ascites with S-180 cell suspension was drawn out from the abdomen of the third passage mouse, and germ-free physiological saline solution was added. The final concentration of the cell suspension was 1×10⁷ cells/ml. 0.1 ml of the cell suspension was injected subcutaneously in the right armpit of the mouse to grow a solid tumor. Four days later, a small mass was seen and palpated on every implanted mouse armpit.

To prepare the mice for the experiment, the hair on the tumor area of the mice was removed with depilatory cream. Then 20 mg/Kg of SF1 was injected into the mouse abdomen cavity in a dark room. Six hours after the injection, some of the tumor-bearing mice were partially submerged in water. A probe manufactured by Angel Ultrasound was then placed into the water to irradiate the tumor area at different intensities for three minutes.

### Results

A. The Inhibitive Effect of SDT with SF1 on S-180 Sarcoma in Mice

1. Comparison of the Tumor Weight in Each Group at the End of this Study:

The tumor bearing mice were allocated into 4 groups with 5 mice in each group: 1) sham-treatment (control, C); 2) ultrasound treatment (only ultrasound treatment, 1.2 W/cm², 1 MHz, without SF1, U); 3) SF1-treatment (SF1 20 mg/Kg ip without ultrasound treatment, S); 4) SF1+ultrasound treatment (S 20 mg/Kg ip+U 1.2 W/cm² and 1 MHz, SU). Fifteen days later, the mice in the four groups were all sacrificed. The tumors of the mice were separated and weighted.

TABLE 1
Tumor weight in each group 15 days after treatment

| | Mean of tumor | P (Comparing with |
|---|---|---|
| group | weight (g) | C) |
| C | 0.361 ± 0.094 | |
| U | 0.440 ± 0.275 | >0.05 |
| S | 0.272 ± 0.328 | >0.05 |
| SU | 0.009 ± 0.003 | <0.01 |

Comparing with group C (control), the tumor weight in group SU was significantly lower (P<0.01). The tumor weight in group U and S had no significant difference like that of group C. This demonstrated that the SF1 plus sound treatment inhibited S-180 sarcoma in mice.

2. Comparison of the Growth Curves in Each Group:

After treatment with SDT, primary tumor size were estimated by measuring perpendicular minor dimension (W) and major dimension (L) using sliding calipers every one or two days. Approximate tumor volume was calculated by the formula: W²L×½.

The tumor in the control group continued to grow. The tumors in groups U and S grew slightly slower than that in group C. But as Table 2 shows, the end sizes in groups U and S were not significantly different from the control group. In group SU, some of the tumors enlarged after SDT, but seven days later, the tumors in all the five mice gradually shrunk. At the end, their size was even smaller than that of before SDT therapy. These results demonstrated again that SDT with SF1 did inhibit the growth of S-180 sarcoma in mice. The data also suggests that SDT not only destroyed the tumors, but also caused inflammatory reaction in the tumor area during the first seven days.

TABLE 2
Tumor size in each group 15 days after treatment

| | Mean of tumor | P (Comparing |
|---|---|---|
| Group | size (cm³) | with C) |
| C | 0.865 ± 0.124 | |
| U | 0.799 ± 0.315 | >0.05 |
| S | 0.611 ± 0.190 | >0.05 |
| SU | 0.047 ± 0.019 | <0.01 |

### B. The Influence of Sound Intensity on SDT Therapeutic Effect to S-180 Sarcoma in Mice

1. Comparison of the Tumor Weight in Each Group at the End of this Study:

The tumor-bearing mice were allocated into four groups with five mice in each group: 1) sham-treatment (control, C); 2) S+0.3 W/cm², 1 MHz ultrasound treatment, SU1); 3) S+0.6 W/cm², 1 MHz ultrasound treatment, SU2); 4) S+1.2 W/cm², 1 MHz ultrasound treatment, SU3). Here S means ip injection with SF1 20 mg/Kg as described in materials and methods. Fifteen days later, the mice in the four groups were all sacrificed and the tumors of the mice were peeled off and weighed.

As Table 3 shows, the tumor weight in the three SDT treated groups was much lower than that in Control group (P<0.05). These results conform to the conclusion that SDT with SF1 inhibits S-180 sarcoma in mice. The tumor weights within the SDT groups also had statistically significant differences between group SU3 and SU1 (P<0.01). It is very clear that the higher intensity of ultrasound used, the higher inhibitive response was produced at the range of ultrasound intensity from 0.3 W/cm² to 1.2 W/cm².

TABLE 3
Tumor weight in each group 15 days after treatment

| | Mean of | P | P |
|---|---|---|---|
| | tumor weight | (Comparing | (Comparing |
| group | (g) | with Control) | with SU1) |
| Control | 0.361 ± 0.094 | | <0.05 |
| SU1 | 0.0425 ± 0.025 | <0.05 | |
| SU2 | 0.021 ± 0.006 | <0.01 | <0.05 |
| SU3 | 0.009 ± 0.003 | <0.01 | <0.01 |

2. Comparison of Tumor Growth Curves in Each Group:

After treatment with SDT, the tumor size was measured with sliding calipers every one or two days. The results are shown in Table 4.

TABLE 4
the tumor size in each group 15 days after treatment

| | Mean of tumor size | P (Comparing with |
|---|---|---|
| group | (cm³) | SU1) |
| Control | 0.865 ± 0.124 | <0.05 |
| SU1 | 0.383 ± 0.113 | |
| SU2 | 0.118 ± 0.020 | <0.05 |
| SU3 | 0.047 ± 0.019 | <0.01 |

As Table 4 shows, the tumors in the 3 SDT treatment groups were much smaller than that in Control group (P<0.05). The tumor weights within the SDT groups also had statistically significant differences between group SU3 and SU1 (P<0.01). Further, in comparison with the control group, the tumor in group SU1 grew much slower, but seven days later it still continued to enlarge. In group SU2 and SU3, the tumors had stopped growing about five to seven days after SDT treatment and then began to shrink. The tumor in group SU3 shrank faster than the tumor in group SU2. Obviously, the inhibitive effect of SDT with SF1 was sound intensity dependent.

The pathological study results in group SU (SF1 20 mg/Kg and ultrasound of 1.2 W/cm²) also showed superior results. Pathological slices were made from the mice sacrificed at 2 hrs, 36 hrs and 15 days after SDT. Coagulated necrosis or metamorphic tissue with inflammatory reaction in the tumor was observed and that the processes of necroses, degeneration and inflammation were further enhanced 36 hrs after the SDT treatment. Fifteen days after SDT, only coagulated necroses and vacuole degeneration was visible in the tumor, but no living tumor cells could be identified. There was some inflammation and fibrosis around the necrotic or degenerative tumor. These data revealed that SDT with SF1 destroyed the S-180 sarcoma mouse very rapidly. The degeneration of tumor induced by SDT occurred almost immediately or at least within two hours after SDT treatment. These data also revealed that along with the necroses and degeneration of the tumor, SDT also induced inflammatory reaction in the tumor and the reaction may last for seven days.

Observations with confocal laser scanning microscopy suggest that SF1 accumulates specifically within tumor cells.

C. SDT with a Piece of Bone Between Tumor and Ultrasound

The tumor-bearing mice were allocated into 2 groups: 1) control treatment (control, C) group; 2) SF1 and ultrasound treatment (SU); the mice in SU group were injected intra-peritoneally with 20 mg/Kg of SF1. Six hours later, the mice were put into the water, covered with a piece of dog thigh bone with an average thickness of 3 mm, and then irradiated with an ultrasound of 2 W/cm² and 1 MHz through the piece of bone to the tumor area for six minutes. 15 days later the tumors were peeled off from each group of mice and weighted.

TABLE 5
Tumor weight in each group 15 days after treatment

| | Mean of tumor | P (Comparing with |
|---|---|---|
| group | weight (g) | C) |
| C | 0.73466 ± 0.0781 | |
| SU | 0.07416 ± 0.0158 | >0.01 |

SDT with a piece of bone between tumor and ultrasound was still able to inhibit the tumor growth. This revealed that 1 MHz ultrasound can pass through bone, activate the sensitizer in the tumor and lead to tumor destruction.

D. The Inhibitive Effect of SDT with Different Ultrasound Frequency on S-180 Sarcoma in Mice

The tumor-bearing mice were allocated into four groups with more than five mice in each group: 1) control (control, C); 2) S+1 W/cm², 0.5 MHz ultrasound treatment, SU1); 3) S+1 W/cm² and 1 MHz ultrasound treatment, SU2); 4) S+1 W/cm² and 2.5 MHz ultrasound treatment, SU3). Here S means ip injection with SF1 20 mg/Kg as described in materials and methods. Following treatment, tumor volume was monitored. Eight days later, the mice in the four groups were all sacrificed and the tumors of the mice were peeled off and weighed.

TABLE 6
Tumor weight in each group 8 days after treatment

| | Mean of tumor | P (Comparing with | P (Comparing with |
|---|---|---|---|
| group | weight (g) | C) | SU2) |
| C | 0.43208 ± 0.128413 | | |
| SU1 | 0.12515 ± 0.019856 | <0.01 | >0.05 |
| SU2 | 0.111967 ± 0.031018 | <0.01 | |
| SU3 | 0.121633 ± 0.020449 | <0.01 | >0.05 |

Comparing with group C (sham treatment), the tumor weight in every SDT treated groups was significantly lower (P<0.01). This demonstrated again that the SF1 plus sound treatment did inhibit S-180 sarcoma in mice. But the tumor weight in group SU1, SU2 and SU3 had no significant difference. The data suggests that 0.5 to 2.5 MHz ultrasounds were all able to active SF1 and destroy the tumor.

### Example 4

### Activated Therapy (Sonodynamic and Photodynamic Therapy (SPDT))

A dose of 45 mg of photo/sonosensitizer is administered sublingually over 2 to 5 hours. No photosensitivity from normal ambient light, artificial or natural has been noted but as a precaution patients are advised not to stay in direct sunlight for periods over half an hour for one week following sensitizer administration. After 48 hours the patient is then exposed to a light bed containing 48 panels, each with 1028 LED's emitting a combination of visible and infra-red light at the frequencies 630 nm and 820 nm. Light bed exposure varies from two sessions of 2 to 15 minutes per day with shorter exposure duration in cases with larger tumor load. Ultrasound is applied using a single maniple at 1 W/cm2 and a frequency of 1 MHz at sites of known malignant disease for 10-30 minutes total. Light and ultrasound activation is repeated on three consecutive days. Ozone auto-haemotherapy (40 IU) is administered immediately before light bed exposure. Further the sensitizer is usually administered after one week for a second treatment cycle. Dexamethasone is administered to some patients with significant tumour load, with dosage titrated on a case by case basis

While this invention has been particularly shown and described with references to preferred embodiments thereof, it will be understood by those skilled in the art that various changes in form and details may be made therein without departing from the scope of the invention encompassed by the appended claims.

1. A method for activated therapy comprising administering to a subject in need thereof a compound having formula I: 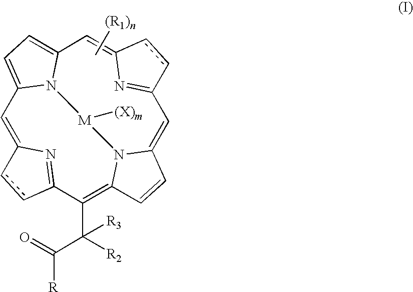
or a pharmaceutically acceptable salt, ester or prodrug thereof, wherein R is OR₄ or NR₄R₅ and each R₄ and R₅ is independently selected from hydrogen, a substituted or unsubstituted, saturated or unsaturated alkyl group or a substituted or unsubstituted aryl group; alternatively, R₄ and R₅ can be taken together with the nitrogen they are attached to form a substituted or unsubstituted heterocyclic group;
each R₁ is independently selected from a substituted or unsubstituted, saturated or unsaturated alkyl group, a substituted or unsubstituted aryl group, acid, ester, amide, amine, substituted amine, acyl, hydroxy, ether, halogen, nitrile, aldehyde, thiol, thioether, sulfonic acid, sulfonate, sulfonamide, and sulfate;
R₂ and R₃ are independently selected from hydrogen, a substituted or unsubstituted, saturated or unsaturated alkyl group;
n is zero or an integer from 1 to 10; [symbol]
represents a single or double bond;
M represents a metal at oxidation state I-VII;
X is an anion or is selected from the group consisting of F, Cl, Br, I, H, CN, a substituted or unsubstituted hydroxide group, a substituted or unsubstituted amino group, a substituted or unsubstituted, straight or branched C₁-C₂₀ alkyl group, an acyl group, a thiolate group or a dialkylamino group; and
m represents 0, 2, 3, 4 or 5 and is chosen to maintain the electric neutrality of the metal complex compound; and
subjecting the individual to ultrasound or red light.

2. The method of claim 1 wherein the activated therapy is sonodynamic therapy, photodynamic therapy or a combination thereof.

3. The method of claim 1, wherein R₂ and R₃ are hydrogen.

4. The method of claim 3, wherein R is OH or NR₄R₅, and R₄ is hydrogen and R₅ is a substituted alkyl.

5. The method of claim 1, wherein the compound has the formula (II): 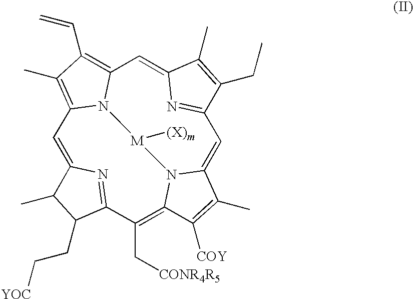
wherein Y is hydroxy, substituted hydroxy, prodrug group or an acceptable metal salt.

6. The method of claim 5, wherein the M is a metal at oxidation state IV and m is 2.

7. The method of claim 6, wherein the metal is Sn(IV).

8. The method of claim 7, wherein R₄ is hydrogen and R₅ is a —(CH₂CH₂O)rCH₂CH₂OH wherein r is an integer between 1 and 100.

9. The method of claim 7, wherein NR₄R₅ is an amino acid, amino acid derivative or peptide.

10. The method of claim 9, wherein NR₄R₅ is an amino acid.

11. The method of claim 1, wherein the compound has the formula III: 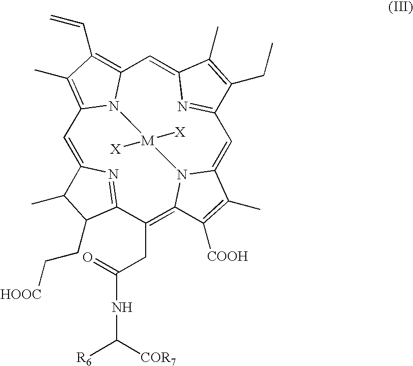
or a pharmaceutically acceptable salt, ester or prodrug thereof, wherein R₆ is hydrogen, a substituted or unsubstituted, saturated or unsaturated alkyl, substituted or unsubstituted aryl;
R₇ and is a hydroxy, substituted hydroxy, amine or substituted amine;
M and X are as previously defined in claim 1.

12. The method of claim 11, wherein R₆ is a hydrogen, C₁-C₅ alkyl, hydroxy C₁-C₅ alkyl, or amino-C₁-C₅ alkyl.

13. The method of claim 12, wherein R₆ is aminobutyl, R₇ is hydroxy.

14. The method of claim 12, wherein R₆ is hydroxymethyl, R₇ is hydroxy.

15. The method of claim 12, wherein R₆ is hydrogen, R₇ is polyglycine.

16. The method of claim 5, wherein R₄ is hydrogen and R₅ is folate.

17. The method of claim 1, wherein each X is hydroxide or acetate.

18. The method according to claim 17 for therapy of cancer.

19. The method according to claim 18, wherein the cancer is a melanoma, colon, breast, lung, or prostate cancer melanoma, prostate, brain, colon, ovarian, breast, skin, lung, esophagus or bladder cancer.

20. The method according to claim 17 for treating a bacterial, viral or parasitic infection.

21. The method according to claim 17 for treating a cardiovascular disease or a neurodegenerative disease.

22. A pharmaceutical composition comprising a compound of formula I as set forth in claim 1, and a pharmaceutically acceptable carrier.
